(19) United States
(12) Patent Application Publication
(10) Pub. No.: US 2025/0369888 A1
(43) Pub. Date: Dec. 4, 2025

(54) RAMAN-BASED QUALITY MONITORING OF BIOPHARMACEUTICAL PRODUCTION PROCESSES

(71) Applicant: THERMO SCIENTIFIC PORTABLE ANALYTICAL INSTRUMENTS INC., TEWKSBURY, MA (US)

(72) Inventors: Nimesh Khadka, Dracut, MA (US); Kristina Pleitt, Cottleville, MO (US); Michelle Nolasco Rivera, St. Louis, MO (US)

(21) Appl. No.: 19/222,826

(22) Filed: May 29, 2025

Related U.S. Application Data
(60) Provisional application No. 63/654,652, filed on May 31, 2024.

(51) Int. Cl. G01N 21/65; G01N 33/68; G16C 20/20; G16C 20/70

(52) U.S. Cl. CPC G01N 21/65; G01N 33/6803; G16C 20/20; G16C 20/70; G01N 2201/1293

(57) ABSTRACT

Software and hardware that can be used to perform quality control at various stages of a biopharmaceutical production process, e.g., during upstream or downstream processing. In some examples, the disclosed Raman-based solutions enable real-time or near real-time quantification of protein concentration in various units of the biopharmaceutical production equipment, including but not limited to bioreactors, product holding vessels, and fluid-transfer lines. In some other examples, the disclosed Raman-based solutions enable real-time or near real-time elucidation and monitoring of the secondary structure of the protein, as a quality marker. In at least some examples, the equipment includes an electronic controller configured to perform or initiate an equipment- or process-control action based on the concentration measurements and/or evaluation of the secondary structure.

### RELATED APPLICATIONS

This application claims priority to U.S. Provisional Application No. 63/654,652, filed May 31, 2024, the entire contents of which is incorporated herein by reference.

### TECHNICAL FIELD

Various examples relate generally, but not exclusively, to methods and apparatus for monitoring and controlling one or more attributes or parameters in a biopharmaceutical production process.

### BACKGROUND

Raman spectroscopy is a spectroscopic technique used to measure the intensity and wavelengths of light in elastically scattered from analytes. A source of monochromatic light, usually a laser emitting in the visible, near infrared, or near ultraviolet spectral range, is used to illuminate the sample. The laser light interacts with molecular vibrations, phonons, and/or other excitations in the sample, resulting in the energy of the laser photons being shifted up or down. The shifts in energy are measured with a spectrometer to obtain a Raman spectrum of the sample. The Raman spectrum can then be analyzed, e.g., to determine certain characteristics of the sample.

### SUMMARY

Disclosed herein are, among other things, various examples, aspects, features, and embodiments of software and hardware that can be used to perform quality control operations at various stages of a biopharmaceutical production process during upstream and downstream processing. In some examples, the disclosed Raman-based solutions enable real-time or near real-time quantification of protein concentration in various units of the biopharmaceutical production equipment, including but not limited to bioreactors, product holding vessels, and fluid-transfer lines. In some other examples, the disclosed Raman-based solutions enable real-time or near real-time elucidation and monitoring of the secondary structure of the protein, as a quality marker. In at least some examples, the equipment includes an electronic controller configured to perform or initiate an equipment- or process-control action based on the concentration measurements and/or evaluation of the secondary structure.

In one example, a method performed via a computing device for providing support to a Raman spectrometry (RS) system comprises: receiving from the RS system a set of electrical readout signals representing a Raman spectrum of a sample including a protein; applying a set of preprocessing operations to the Raman spectrum to obtain a corresponding preprocessed Raman spectrum conforming to an input format of a selected multivariate chemometric model; and estimating a concentration of the protein in the sample using the selected multivariate chemometric model and the preprocessed Raman spectrum.

In another example, a method performed via a computing device for providing support to an RS system comprises: receiving from the RS system a set of electrical readout signals representing a first Raman spectrum and a second Raman spectrum of first and second samples, respectively, including a protein in different respective concentrations; applying a set of preprocessing operations to the first and second Raman spectra to obtain a corresponding difference spectrum; and analyzing a selected spectral portion of the difference spectrum to evaluate a secondary structure of the protein.

According to yet another example, provided is a non-transitory computer-readable medium storing instructions that, when executed by the computing device, cause the computing device to perform operations comprising any one of the above methods.

In one example, an apparatus comprises: an RS system; and a computing device configured to: receive from the RS system a set of electrical readout signals representing a Raman spectrum of a sample including a protein; apply a set of preprocessing operations to the Raman spectrum to obtain a corresponding preprocessed Raman spectrum conforming to an input format of a selected multivariate chemometric model; and estimate a concentration of the protein in the sample using the selected multivariate chemometric model and the preprocessed Raman spectrum.

In another example, an apparatus comprises: an RS system; and a computing device configured to: receive from the RS system a set of electrical readout signals representing a first Raman spectrum and a second Raman spectrum of first and second samples, respectively, including a protein in different respective concentrations; apply a set of preprocessing operations to the first and second Raman spectra to obtain a corresponding difference spectrum; and analyze a selected spectral portion of the difference spectrum to evaluate a secondary structure of the protein.

### BRIEF DESCRIPTION OF THE DRAWINGS

The foregoing aspects and many of the attendant advantages of the present disclosure will become more readily appreciated as the same become better understood by reference to the following detailed description, when taken in conjunction with the accompanying drawings.

FIG. 1 is a block diagram illustrating a biopharmaceutical production process according to some examples.

FIG. 2 is a block diagram illustrating an ultrafiltration/diafiltration system used in the biopharmaceutical production process of FIG. 1 according to one example.

FIG. 3 is a diagram illustrating a cross-sectional side view of a Raman probe used for quality monitoring in the biopharmaceutical production process of FIG. 1 according to some examples.

FIG. 4 is a block diagram illustrating a Raman instrument used with the biopharmaceutical production process of FIG. 1 according to some examples.

FIG. 5 is a flowchart illustrating a method performed via a computing device for providing support to the Raman instrument of FIG. 4 according to some examples.

FIGS. 6A-6C show a table listing responsive actions that can be taken or initiated in the method of FIG. 5 according to some examples.

FIGS. 7-14 graphically illustrate a first chemometric model and its use in the method of FIG. 5 according to some examples.

FIGS. 15-21 graphically illustrate a second chemometric model and its use in the method of FIG. 5 according to some examples.

FIG. 22 graphically illustrates improvements in protein concentration measurements obtained with the method of FIG. 5 according to some examples.

FIG. 23 is a flowchart illustrating a method performed via a computing device for providing support to the Raman instrument of FIG. 4 according to further examples.

FIGS. 24-30 graphically and schematically illustrate certain operations of the method of FIG. 23 according to some examples.

FIG. 31 is a block diagram illustrating a computing device according to some examples.

### DETAILED DESCRIPTION

A monoclonal antibody (mAb) is an antibody produced from a cell lineage made by cloning a unique white blood cell. Monoclonal antibodies typically have monovalent affinity, binding only to the same epitope (the part of an antigen that is recognized by the antibody). In contrast, polyclonal antibodies bind to multiple epitopes and are usually made by several different antibody-secreting plasma cell lineages. It is possible to produce monoclonal antibodies that specifically bind to a selected substance. Those monoclonal antibodies can then be used to detect or purify that substance. In some applications, monoclonal antibodies are used in the diagnosis of illnesses, such as cancer or infections, and are also used therapeutically in the medical treatment of certain diseases.

The number of monoclonal antibodies approved for therapeutic use has been steadily increasing. This increase is due in part to the improvements in the large-scale manufacturing processes that are used to produce large quantities of monoclonal antibodies and other proteins. Efficient recovery and purification of proteins from cell culture media is an important part of the production process. The purification process is designed to produce the end-product proteins that are safe for use in humans. Such purification process typically incorporates quality monitoring that includes, for example, monitoring certain protein attributes and impurities that can potentially impact the patient safety and/or the drug efficacy or potency. Protein concentration is also an important attribute of the purified material, and appropriate protein concentrations in different process intermediates are important process parameters that can affect the unit operation performance.

To ensure that the final formulations of monoclonal antibodies or other proteins meet the applicable specifications and standards, the corresponding bioproducts are tested at various stages of the production process. In some cases, quality control in the manufacturing of bioproducts, such as monoclonal antibodies, is accomplished by analyzing purification intermediates and formulated drug substance samples with offline methods for each lot production. In such cases, the samples are removed from the processing equipment and subjected to offline tests to measure product quality attributes, such as the protein concentration (g/L), buffer excipients, and size variants. Real-time monitoring and analysis during manufacturing is typically more advantageous than the offline methods because it can significantly decrease the processing time and lower the risk of batch failure due to not meeting the applicable specifications. Accordingly, rapid, in-line methods of real-time quality control monitoring of bioproducts are being actively developed in the biopharmaceutical industry.

In at least some production processes, in-line analytics where the measurement occurs in the main fluid stream, on-line analytics where the measurement occurs adjacent to the main fluid stream, and at-line analytics where measurement is performed after sampling beneficially enable process technicians to make decisions in real-time, which beneficially saves production time, cost, and resources. For example, in conventional workflows, after the ultrafiltration/diafiltration (UF/DF) process, the samples are sent for offline analyses to quantify excipient concentration. The offline-analysis results may become available within hours to days, which oftentimes causes the production technicians to proceed with assumptions based on the theoretical values rather than on actual experimental observations. However, such assumptions may not provide sufficiently accurate data due to various demonstrated phenomena, which may cause non-negligible offsets in concentration values. In addition, the offline-analysis delays typically add cost to the product and also stand as a significant obstacle to moving toward continuous manufacturing. Furthermore, excipient concentrations have gained special importance in the mAb formulation development area as final-product concentrations have shifted to >100 g/L to enable subcutaneous administration to the patients.

In some cases, UV-Vis-based in-line quantification of protein concentrations can be used in addition to or instead of offline measurements. However, for some proteins, several pertinent matrices may overlap with those proteins in the UV-Vis absorption spectra, thereby interfering with the UV-Vis-based analysis and concentration measurements. Other limitations of UV-Vis spectroscopy, e.g., as applied to mAb products and intermediates, may further disadvantageously restrict its applicability in the in-line, on-line, and at-line analytics.

Various embodiments disclosed herein are directed at providing accurate, rapid, real-time or near real-time, cost-saving Raman-based solutions for quantifying proteins, excipients, and aspects of protein quality throughout the production workflow to improve the process observability and control. In some examples, Raman spectra are obtained using flow-through Raman cells designed for easy integration into continuous flow applications as parts of the in-line, on-line, and at-line process analytical technology (PAT). In some other examples, Raman spectra are obtained by interrogating extracted samples of mAb products with at-line or offline Raman instruments. The obtained Raman spectra are mathematically processed to isolate spectral features corresponding to the protein products and intermediates, which are then used, e.g., to quantify protein concentrations at various stages of the production process during upstream and downstream unit operations.

In some examples, various disclosed Raman-based solutions are capable of providing one or more of the following benefits and/or advantages:
(i) Accurate real-time or near real-time quantification of protein concentration during upstream and downstream purification processes.
(ii) Accurate protein quantification in a variety of matrices, including the matrices containing components that interfere with UV-Vis analyses, such as aromatic amino acids, contaminant proteins, surfactants, and the like.
(iii) Accurate quantification of excipient concentration during the downstream processes, e.g., UF/DF with histidine, arginine, and sucrose.
(iv) Elucidation of the protein secondary structure in real-time, e.g., as a quality marker.
(v) Real-time or near real-time monitoring of the protein quality via the attributes including but not limited to disulfide bond intactness, protein conjugation chemistries, glycosylation ratio, glycan ratio, and oxidation-reduction chemistries.
   In some examples, the corresponding chemometric models are transferable across different probes, different probe formats, different processes, different unit operations, and different monoclonal antibodies or proteins. In some examples, such models can be integrated into commercial PAT software. In some examples, customers are provided with an option to use pre-developed models to monitor and control their processes or to develop their own chemometric models using the core methodology disclosed herein. In some cases, a similar approach is used for other biomolecules, such as peptide/proteins, L- and D isoforms, nanobodies, Affitins (commercial name Nanofitins), nucleic acids, etc.

As used herein, the term “real time” refers to a computer-based process that controls or monitors a corresponding environment by receiving data, processing the received data, and generating a response sufficiently quickly to affect or characterize the environment without significant delay. In the context of control or processing software, real-time responses are often understood to be on the order of milliseconds, or sometimes microseconds. In the context of a biopharmaceutical production process, “real-time” updates mean that the experimental data and measurement results derived therefrom sufficiently accurately represent the state of the system, product, or intermediate at any point in time. In this case, data-acquisition and/or processing delays of several minutes may still be considered to be within “real time” or “near real time” for at least some processes.

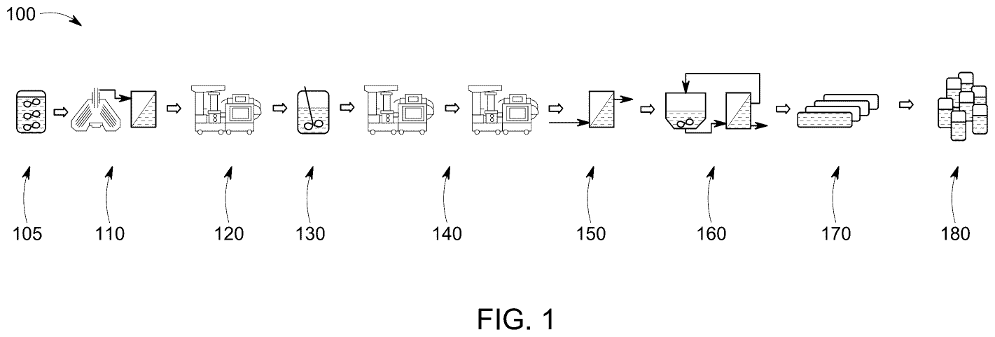

FIG. 1 is a block diagram illustrating a biopharmaceutical production process 100 according to some examples. The process 100 includes upstream and downstream processing (USP and DSP, respectively). Together, USP and DSP are configured to lead to a safe, high-quality end product.

USP is the first part of the biopharmaceutical production process 100. At the USP stage, engineered cell lines, either microbial or mammalian, are utilized for efficient scalable production of the target protein or active pharmaceutical ingredient (API). In a typical example, the USP includes fermentation and harvest, including all stages of cell cultivation, from early cell isolation, media development and preparation, inoculum development, cell banking and storage, all the way to harvest, and the product is collected. A main aim of the USP is to optimize the growth of the production cell line in industrial volumes and settings, thereby leading to the production of large quantities of the target product.

For the production of biopharmaceuticals, microbial cultures or mammalian cells are typically used. Microbial cultures are more appropriate for producing small molecules, such as peptides and enzymes. Production of big, complex proteins, such as monoclonal antibodies, is hindered by their lack of glycosylation mechanism, which is present in mammalian cells. This characteristic makes mammalian cell expression systems a preferred choice for the USP associated with the production of monoclonal antibodies.

After the target product is produced in the cell line expression system, the medium containing the product and cells is harvested. The harvest is then processed in primary recovery as a first step of the purification process. In some examples, this step includes fast separation of the target protein from the bioreactor medium, cells, and cell debris. In general, proteins can be produced intracellularly or extracellularly, which influences the downstream purification steps. In the case of mammalian cell culture, most of the protein is produced extracellularly. This means that only the supernatant needs to be collected for further purification and product concentration. For this purpose, the process 100 includes operating a bioreactor or production vessel 105 and then block 110 that includes, inter alia, centrifugation and microfiltration operations. In various examples, the operations of the block 110 aim to remove most of the water, medium, and small molecules by product concentration. Also, due to the removal of the bioreactor medium, the product is protected from degradation by minimizing proteolytic enzymes. The block 110 is typically considered to be the last stage of the USP. After the block 110, a multi-step purification process starts, which is considered to be a part of the DSP.

In the process 100, the DSP includes blocks 120-180. After the largest impurities are removed in the block 110, the product is subjected to a series of purification steps. Operations of the block 120 include capture chromatography where the target product is isolated and concentrated.

The following types of chromatography may be used in various examples of the block 120: affinity chromatography, ion-exchange chromatography, mixed-mode chromatography, hydrophobic chromatography, and gel-filtration chromatography. Operations of the block 130 include viral inactivation. Operations of the block 140 include intermediate and polishing chromatography. Intermediate purification aims at removing most of the bulk impurities, such as other proteins, nucleic acids, and endotoxins, and at further concentrating the product. For this purpose, ion-exchange chromatography is typically used. A key objective of polishing is to remove trace amounts of impurities remaining. In some examples, size exclusion chromatography (SEC) is used for this purpose. Operations of the block 150 include viral filtration. In some examples, the viral filtration is implemented using ultrafiltration in tangential flow or normal flow, which makes sure that all remaining impurities are removed, including any mammalian infecting viruses and bacterial pathogens and their endotoxins. Operations of the block 160 include the above-mentioned UF/DF process. Operations of the block 170 include bulk fill operations, during which the product is transferred into relatively large flexible or rigid containers configured for intermediate storage and provided with various transfer assemblies to allow for a combination of sizes, flow paths, and other variations. Operations of the block 180 include final fill operations, during which the product is transferred from bulk-fill containers into dispense containers.

In various examples, one or more Raman probes are incorporated into or coupled to the equipment used to implement various operations of the blocks 110-180 of the biopharmaceutical production process 100. For illustration purposes and without any implied limitations, some illustrative examples are described below in reference to using such Raman probes in the block 160 of the process 100. Based on the provided description, a person of ordinary skill in the pertinent art will be able to make and use other examples, in which one or more Raman probes are coupled to or incorporated into the equipment corresponding to the blocks 110-150 and 170-180 of the process 100, without any undue experimentation.

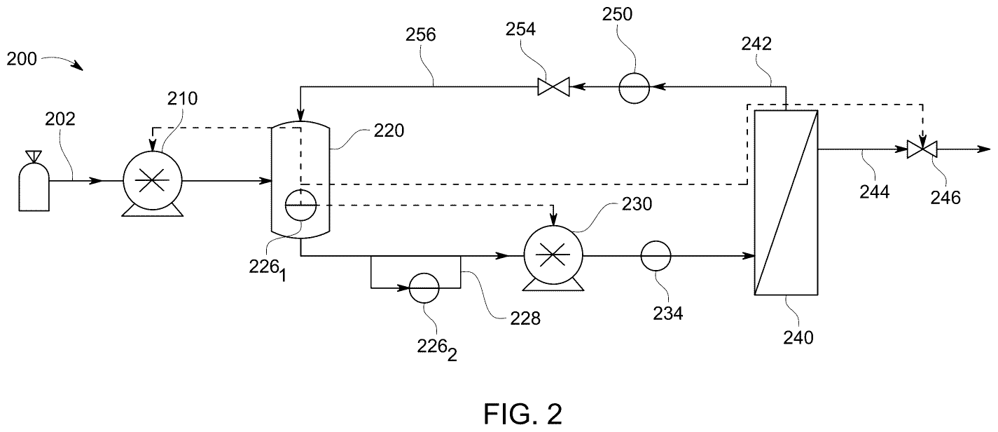

FIG. 2 is a block diagram illustrating a UF/DF system 200, including locations for Raman probe placement, according to one example. In some examples, the UF/DF system 200 is configured to perform some or all operations of the block 160 of the biopharmaceutical production process 100 (FIG. 1).

The system 200 includes a pump 210 configured to pump a protein purification intermediate 202 into a retentate vessel 220. In different examples, the pump 210 can be implemented using a peristaltic, rotary lobe, pressure transfer, centrifuge pump, or diaphragm pump. Fluid from the retentate vessel 220 flows to a feed pump 230 and then, through a feed pressure valve 234, into a tangential flow filtration module (TFFM) 240. In TFFM 240, the protein purification intermediate is subjected to ultrafiltration across a membrane. The bioproduct of interest is retained in a fluid (retentate) 242 while water and low molecular weight solutes including buffer excipients pass through the membrane in a permeate (filtrate) 244 which exits the system 200 by passing through a permeate pressure valve 246. The retentate 242 exits the TFFM 240 and passes through a retentate pressure valve 250, a transmembrane pressure (TMP) control valve 254, and a retentate return channel 256 back into the retentate vessel 220. This circular flow process is repeated as deemed necessary to concentrate the bioproduct, remove impurities, and ensure that the quality attributes and/or parameters are within the acceptable ranges. During diafiltration, the same flow path is followed, with permeable solutes being replaced and a new buffer being washed into the product stream. When the new buffer is added at the same rate as the permeate is removed from the system 200, the sum of the retentate tank volume and the skid hold-up volume defines the system volume. One turn-over volume (TOV) is defined as the amount of diafiltration buffer added to the UF/DF process that is equal to the system volume. Typically, replacement of eight times the system volume (8 TOV) assures a >99.9% buffer exchange.

Additionally, during the UF/DF process performed with the system 200, the protein solution in the retentate vessel 220 is continuously being mixed or agitated. For example, differences in density between the diafiltration buffer, the retentate return, and bulk retentate during diafiltration are addressed with the agitation that is sufficient to ensure adequate buffer exchange, yet sufficiently moderate to avoid shear, as the latter might result in protein aggregation and visible and subvisible particle (SVP) generation in some products. Additionally, it is important to ensure adequate mixing of retentate return during concentration stages to prevent protein concentration polarization in the retentate tank 220 resulting in higher protein concentrations being delivered to the UF/DF membranes in the TFFM 240.

In the example shown, the system 200 includes Raman probes 226₁ and 226₂. In other examples, a different (from two) number of Raman probes 226ₙ can also be used. The Raman probe 226₁ is placed in the retentate vessel 220 and can be an immersible probe. The Raman probe 226₂ is placed in a split-flow branch 228 downstream of the retentate vessel 220 before the feed pump 230 and can be a flow-cell probe. In other examples, additional or alternative Raman probe locations can also be used. For example, in some cases, the Raman probe 226₂ or an additional inline Raman probe 226ₙ is placed between the feed pressure pump 234 and the TFFM 240. In yet some other cases, an additional inline Raman probe 226ₙ is inserted into the retentate return channel 256. In general, a person of ordinary skill in the art will readily recognize what type of probe, e.g., immersible, split flow, or inline, to use and where to place such Raman probes within the system 200 to ensure sufficient and accurate Raman measurements therein.

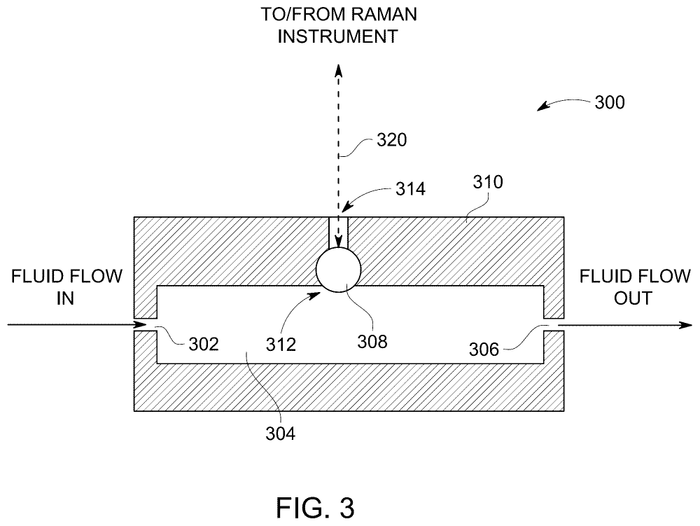

FIG. 3 is a schematic diagram illustrating a cross-sectional side view of a flow-cell Raman probe 300 used with the biopharmaceutical production process 100 according to some examples. In some cases, some of the Raman probes 226ₙ in the system 200 can be implemented using different instances of the Raman probe 300. In various examples, additional instances of the Raman probe 300 can be coupled to or incorporated into other equipment used in the biopharmaceutical production process 100.

The Raman probe 300 includes a flow cell device 310 having a fluid inlet port 302, an inner chamber 304, and a fluid outlet port 306. In operation, a fluid to be interrogated with the corresponding Raman instrument flows from the fluid inlet port 302 into the inner chamber 304 and then out of the inner chamber 304 through the fluid outlet port 306. The flow cell device 310 includes a spherical lens 308 configured to couple light 320 in and out of an analysis zone 312 that is proximate to the lens 308 within the inner chamber 304. The light 320 typically includes the pump (excitation) light and the corresponding scattered light produced in the analysis zone 312. A portion of the surface of the lens 308 forms a part of the wall of the inner chamber 304. Optical coupling between the lens 308 and the corresponding Raman instrument is accomplished via an opening 314 in the body of the flow cell device 310. In some examples, the opening 314 is configured to accept an optical fiber (not explicitly shown) and/or other suitable coupling optics for transmitting the light 320.

In some examples, the Raman probe 300 may benefit from the use of certain features disclosed in U.S. Pat. No. 10,209,176, which is incorporated herein by reference in its entirety. In various examples of the biopharmaceutical production process 100, other suitable Raman probes can also be used. For example, several suitable Raman probes are commercially available under the brand names Thermo Scientific and MarqMetrix.

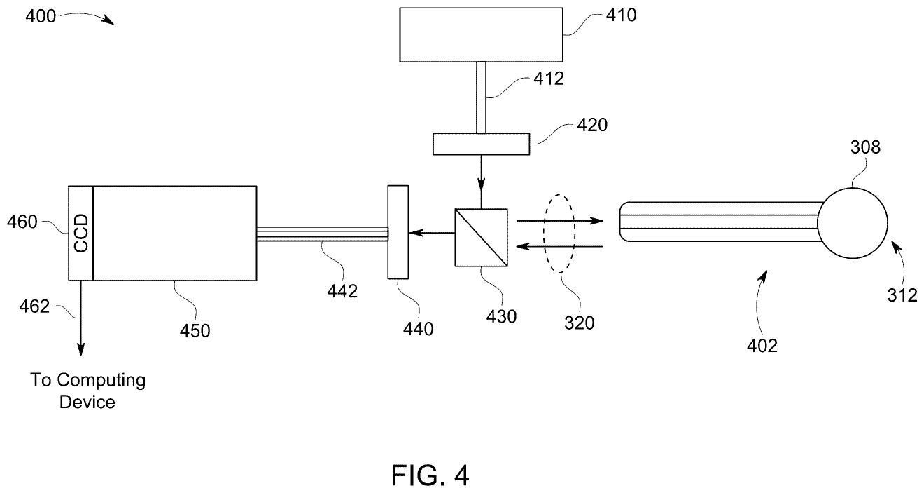

FIG. 4 is a block diagram illustrating a Raman instrument 400 used for quality monitoring in the biopharmaceutical production process 100 according to some examples. For illustration purposes and without any implied limitations, the Raman instrument 400 is shown in FIG. 4 as being optically coupled to an optical fiber probe 402 including the spherical lens 308 (also see FIG. 3). In other examples, the Raman instrument 400 can be optically coupled to other suitable Raman probes (e.g., immersible, flow-cell, inline, at-line, or off-line probes) used for quality control in the biopharmaceutical production process 100 as indicated above.

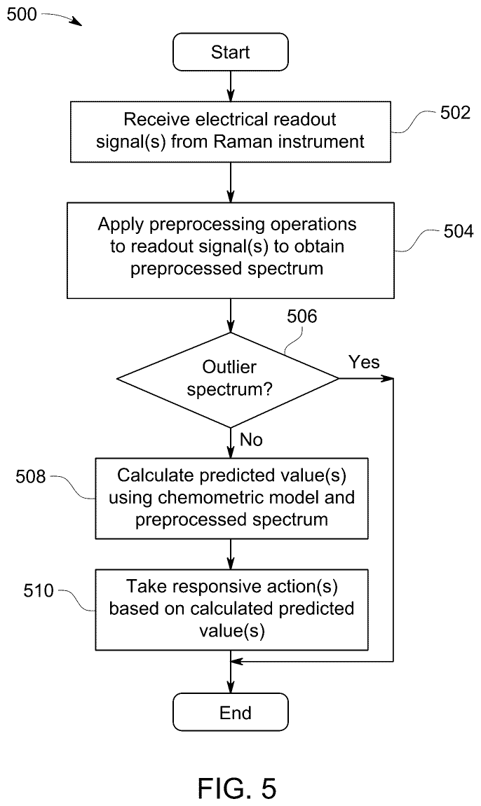

The Raman instrument 400 includes a laser 410, with the optical output thereof being coupled into a first optical fiber 412 that guides the laser light to a narrow bandpass filter 420. A dichroic beam splitter 430 then redirects the laser light filtered with the bandpass filter 420 to the optical fiber probe 402. The scattered light produced in response to the laser light in the analysis zone 312 is collected by the spherical lens 308 and directed via the optical fiber probe 402 back to the dichroic beam splitter 430. The dichroic beam splitter 430 passes through the Raman-scattered light while rejecting (via redirection) most of the elastically scattered laser light. An optical notch filter 440 then substantially fully stops the residual laser light while passing through the Raman-scattered light into a second optical fiber 442. The second optical fiber 442 then guides the received Raman-scattered light to a fiber-coupled spectrometer 450. The spectrometer 450 disperses the received Raman-scattered light in wavelength, and the dispersed light is detected by a pixelated CCD detector 460. Finally, an electrical readout signal 462 from the CCD detector 460 (representing the Raman spectrum of the fluid located in the analysis zone 312) is directed via a communication channel, link, or connection to a corresponding computing device for processing and analysis. Various examples of such processing and analysis are described in more detail below in reference to FIGS. 5-31.

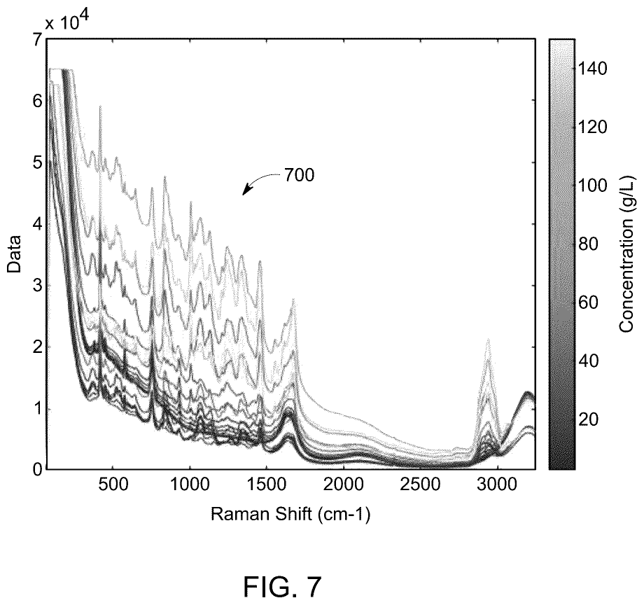

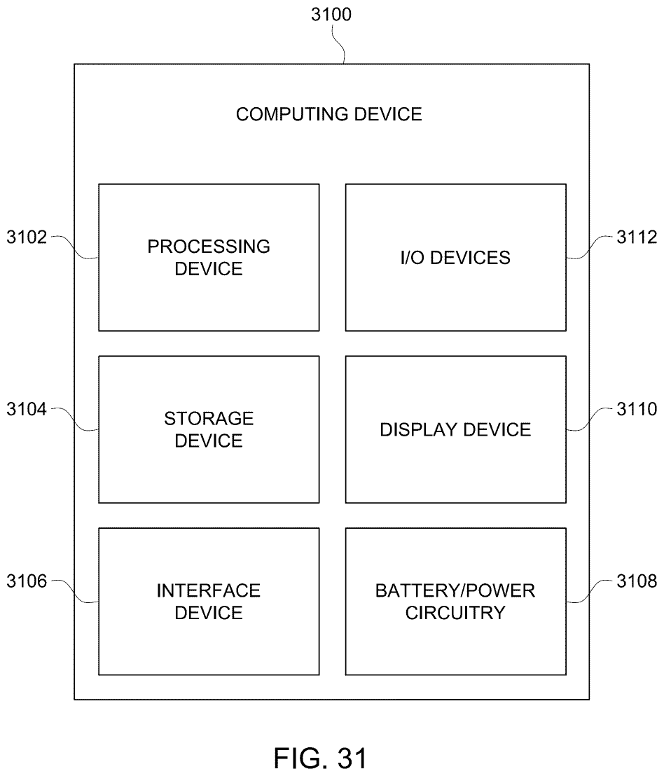

FIG. 5 is a flowchart illustrating a method 500 performed via a computing device for providing support to the Raman instrument 400 according to some examples. In different examples, the method 500 can be configured to use different respective chemometric models. Several example chemometric models that can be used in different implementations of the method 500 are described in more detail below in reference to FIGS. 7-22. An example computing device that can be used to carry out the method 500 is described in more detail below in reference to FIG. 31.

The method 500 includes the computing device receiving from the detector 460 of the Raman instrument 400 one or more electrical readout signals 462 (in a block 502). As already indicated above, each of the readout signals 462 represents a respective Raman spectrum acquired with the Raman instrument 400 from a sample located in, at, or adjacent to a corresponding Raman probe or cell. In some examples, the corresponding Raman probe can be one of the above-described Raman probes 226₁, 226₂, 226ₙ, and 300. In some examples, the corresponding Raman probe can be an immersible, flow-cell, inline, at-line, or off-line probe. In various examples, the set of acquisition parameters with which the Raman instrument 400 performs the measurements corresponding to the received readout signals 462 may be the same as or different from the set of acquisition parameters used to acquire the training data for the chemometric model employed in the method 500. In some examples, the set of acquisition parameters includes: (i) the output wavelength of the laser 410; (ii) the output power of the laser 410; (iii) exposure time of the detector 460 per acquired spectrum; (iv) the number of acquired spectra for averaging; (v) inter-acquisition delay time; and (vi) the flow rate through the flow cell device 310 (when applicable).

The method 500 also includes the computing device applying one or more preprocessing operations (in a block 504) to the readout signal(s) received in the block 502. In various examples, the preprocessing operations of the block 504 may include one or more of the operations selected from the following nonexclusive list: (i) averaging two or more readout signals; (ii) baseline removal; (iii) normalization; (iv) selection of one or more spectral regions that are narrower than the full spectral range covered in the signal acquisition; (v) signal filtering; (vi) computing a derivative; and (vii) mean centering. A result of the preprocessing operations performed in the block 504 is hereafter referred to as a preprocessed spectrum. In various examples, the preprocessed spectrum generated in the block 504 has a format and/or a set of attributes conforming to the input format accepted by the corresponding chemometric model.

The method 500 also includes the computing device determining whether the preprocessed spectrum is an outlier for the chemometric model (in a decision block 506). In one example implementation of the decision block 506, the preprocessed spectrum is projected onto the model space, and the projection is checked for an outlier status using the Q residuals versus Hotelling's T² (Q-v-T) plot. More specifically, if the projection falls within the delineated boundaries on the Q-v-T plot, then the spectrum is judged not to be an outlier. On the other hand, if the projection falls outside such boundaries on the Q-v-T plot, then the spectrum is judged to be an outlier. In other example implementations of the decision block 506, other suitable outlier-determination criteria can also be used in the decision block 506.

When it is determined that the preprocessed spectrum is an outlier (“Yes” at the decision block 506), the spectrum is discarded and the processing of the method 500 is terminated. When it is determined that the preprocessed spectrum is not an outlier (“No” at the decision block 506), the processing of the method 500 advances onto a block 508.

The method 500 also includes the computing device calculating one or more predicted values (in the block 508). The calculations of the block 508 are performed using the preprocessed spectrum and the selected chemometric model. In some examples, the calculated predicted value(s) include the predicted concentration of the corresponding protein(s), e.g., the concentration of monoclonal antibodies at the corresponding quality checkpoint of the process 100. In some examples, operations of the block 508 include: (i) transforming the preprocessed spectrum using the latent variables of the chemometric model to calculate the corresponding score vector and (ii) multiplying the calculated score vector and the regression coefficient vector of the chemometric model to obtain the predicted value(s).

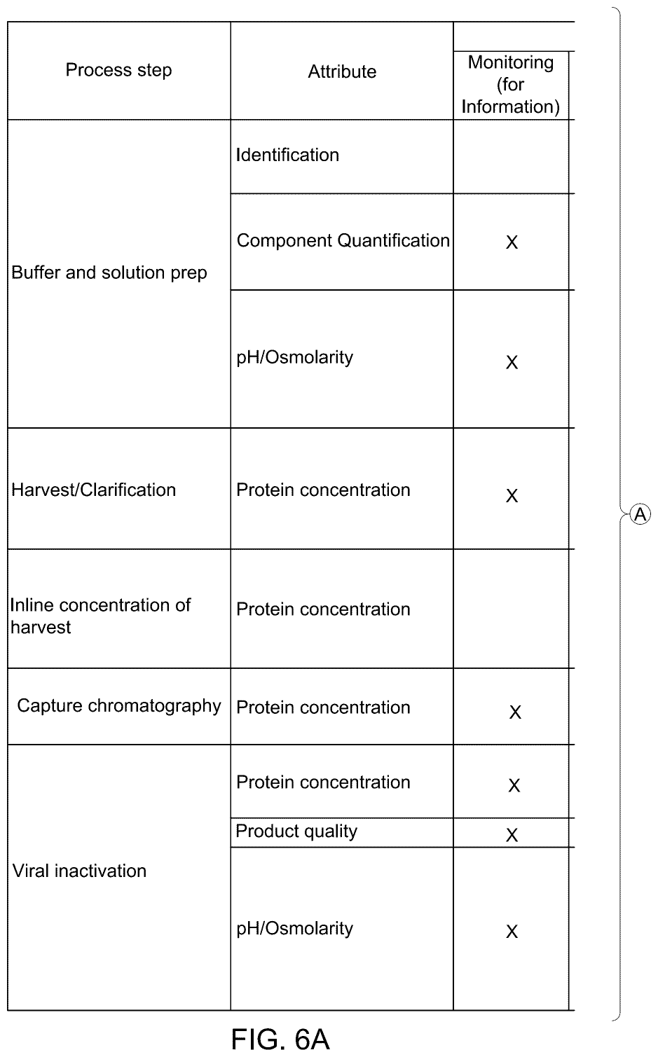

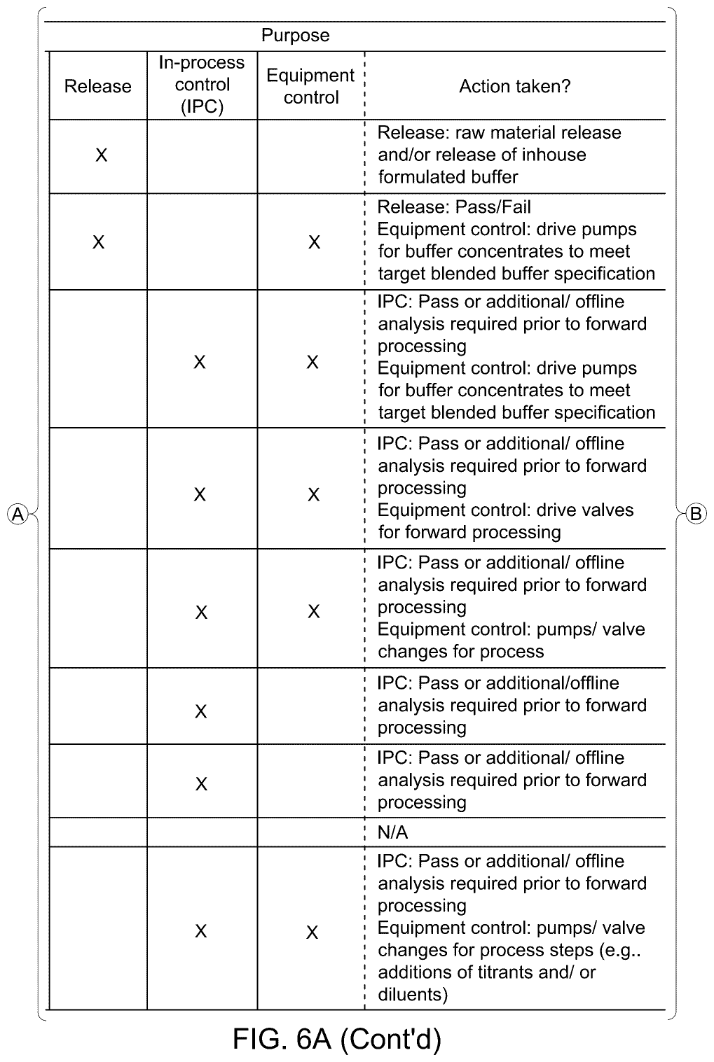

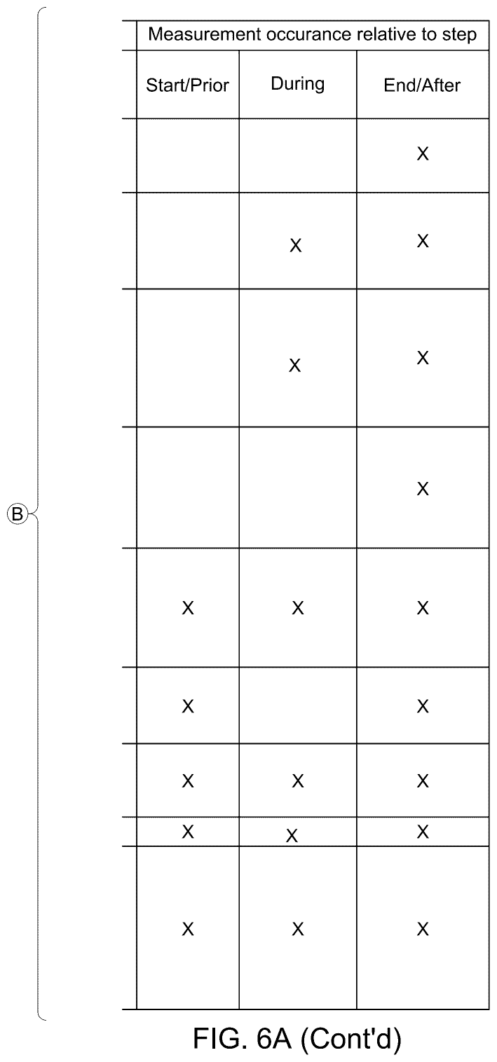

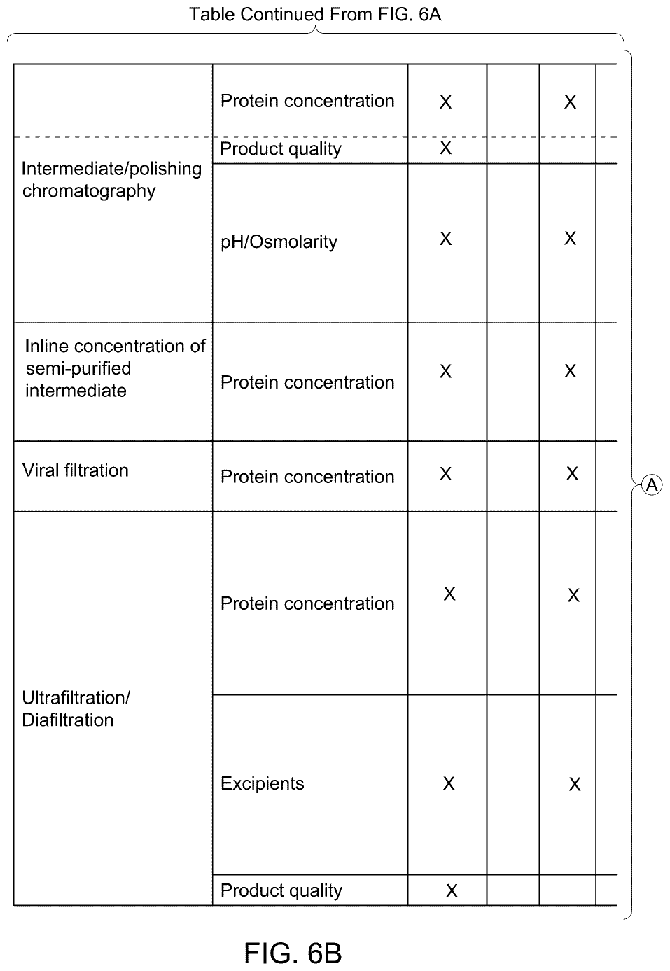

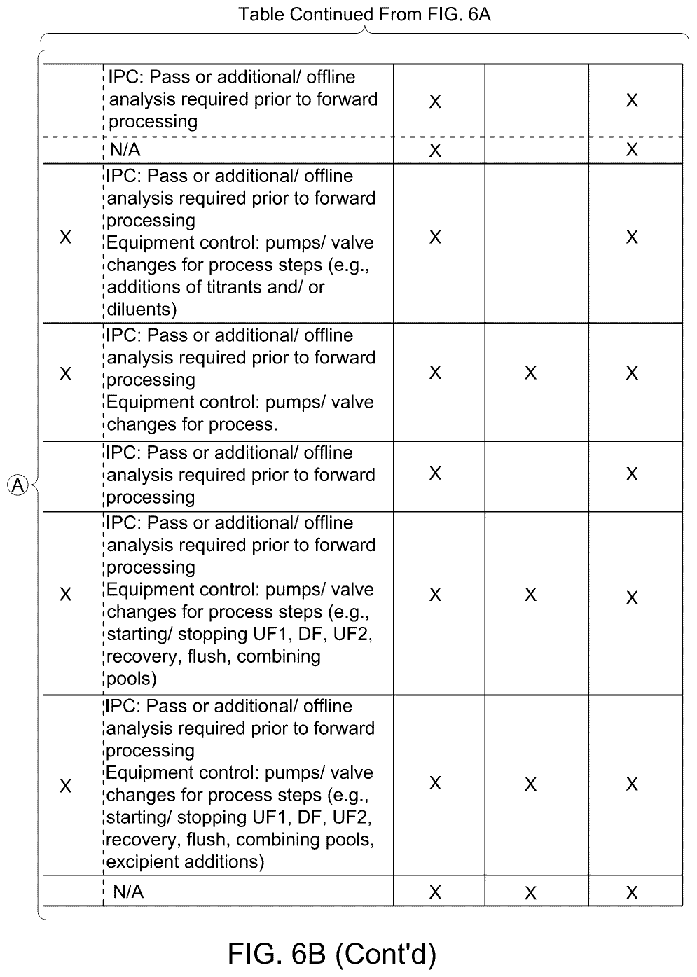

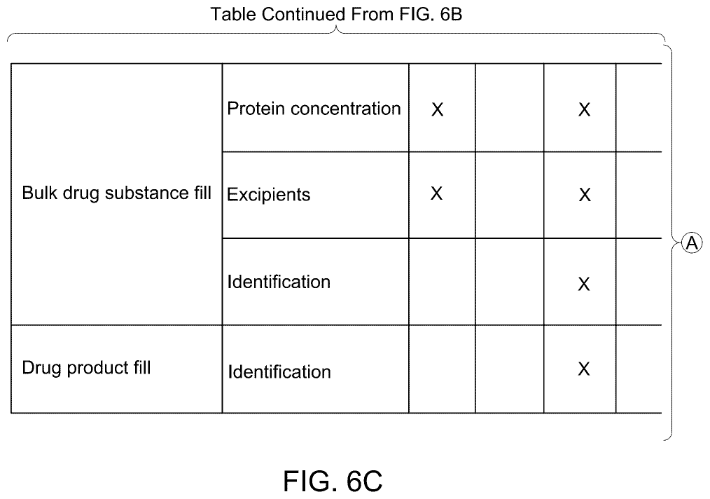

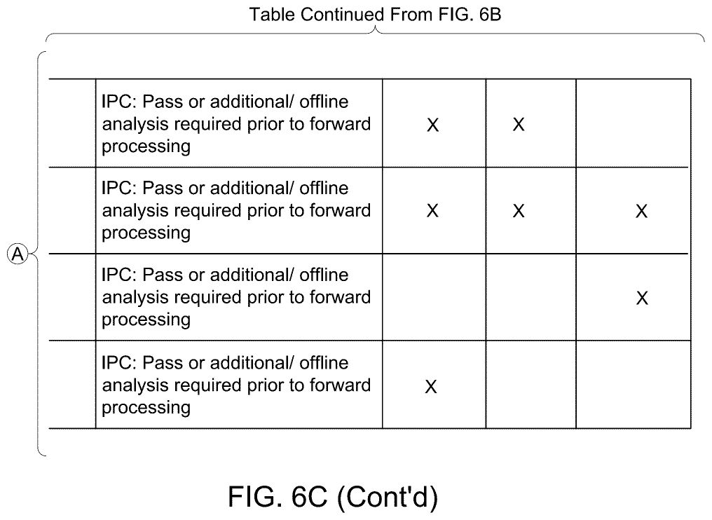

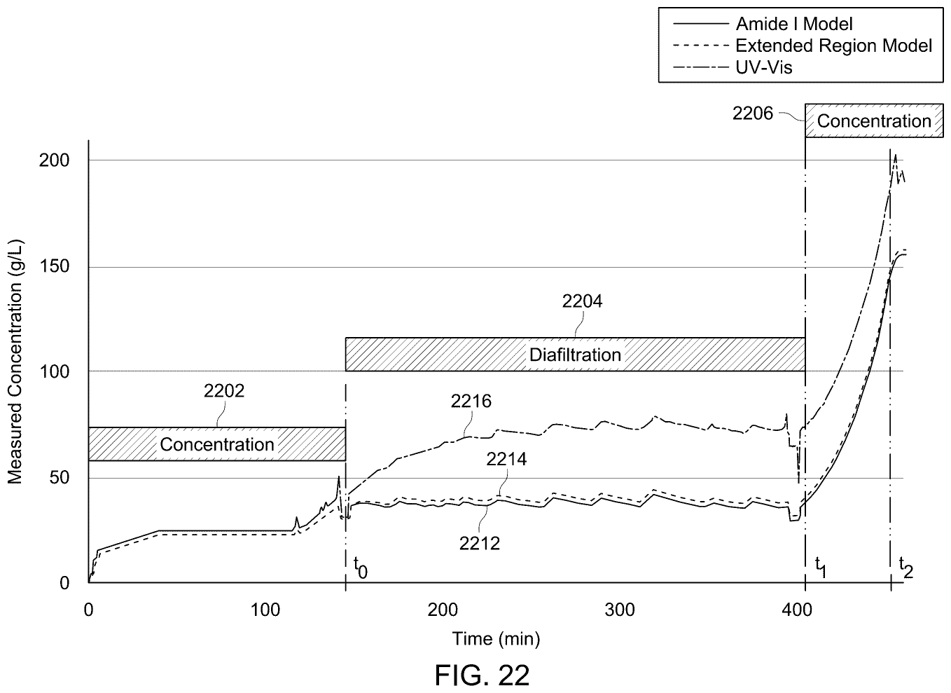

The method 500 also includes the computing device performing or initiating one or more responsive actions (in a block 510). One example of such responsive action includes the computing device displaying a predicted value obtained in the block 508 on a display device. Another example of such responsive action includes the computing device adding the predicted value obtained in the block 508 to a graph or plot shown on a graphical user interface (GUI). In some cases, the displayed plot shows the concentration of monoclonal antibodies as a function of time (e.g., see FIG. 22). Yet another example of such responsive action includes the computing device providing an input, based on the predicted value obtained in the block 508, to a corresponding electronic controller of a component of the equipment used in the process 100 (also see FIG. 6). Upon completion of the operations of the block 510, the method 500 is terminated.

FIGS. 6A-6C show a table listing responsive actions that can be taken or initiated in the block 510 of the method 500 according to some examples. In various examples, a responsive action can be classified as a process-information action, a release action, an in-process control action, or an equipment control action. Some of the responsive actions may fall into two or more classes. For each responsive, action the table presented in FIGS. 6A-6C identifies: (i) the corresponding block, step, or operation of the biopharmaceutical production process 100; (ii) the corresponding attribute(s); (iii) one or more classifiers of the action based on the action's purpose or effect with respect to the biopharmaceutical production process 100; and (iv) the relative timing of the measurement based on which the action is taken.

For illustration purposes and without any implied limitations, example chemometric models are described below in reference to Immunoglobulin G (IgG). IgG is one of the most abundant proteins in human serum, accounting for about 10-20% of plasma protein, and represents one of the five main classes of immunoglobulins in humans. The other classes include IgM, IgD, IgA, and IgE proteins. The IgG class is further divided into four subclasses, namely IgG1, IgG2, IgG3, and IgG4, which are assigned a subclass number in the order of decreasing abundance. Although these subclasses are more than 90% identical at the amino acid level, each subclass has a unique profile with respect to antigen binding, immune complex formation, complement activation, triggering of effector cells, half-life, and placental transport. At least some chemometric models are transferrable between the subclasses in that a chemometric model developed for one subclass provides similarly accurate results when applied to another subclass. Based on the provided description, a person of ordinary skill in the pertinent art will be able to make and use other chemometric models corresponding to other proteins, monoclonal antibodies, classes, and/or subclasses without any undue experimentation.

FIGS. 7-14 graphically illustrate a first chemometric model and its use in the method 500 according to some examples. The first chemometric model makes use of the Raman features spectrally located in the wavenumber range between approximately 1600 cm⁻¹ and approximately 1750 cm⁻¹, which covers Raman bands corresponding to vibrations of the protein's carbonyl group (—CONH) in different secondary structures. These bands are directly relatable to the backbone conformation. In some pertinent literature, this spectral region is referred to as the “Amide I” region. As such, the first chemometric model can also be referred to as the “Amide I” model.

FIG. 7 graphically shows a set 700 of Raman spectra corresponding to different respective concentrations of IgG in the concentration range between 1 g/L and 150 g/L. A set similar to the set 700 can be obtained, e.g., during the model calibration or in one or more instances of the block 502 of the method 500.

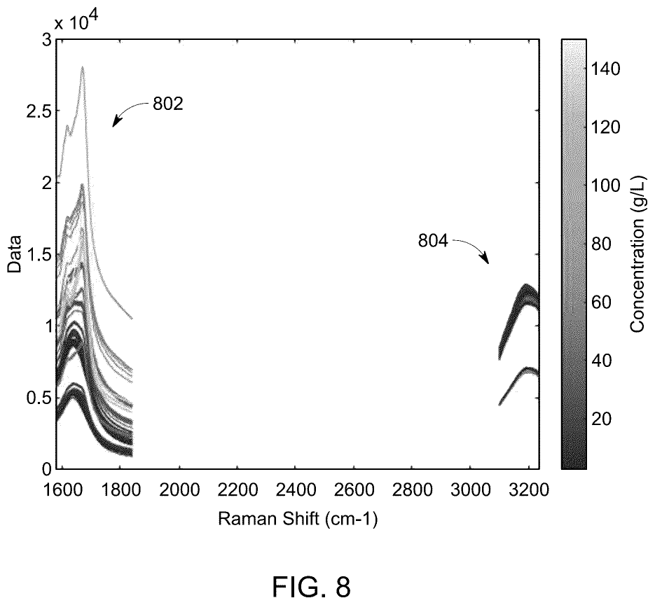

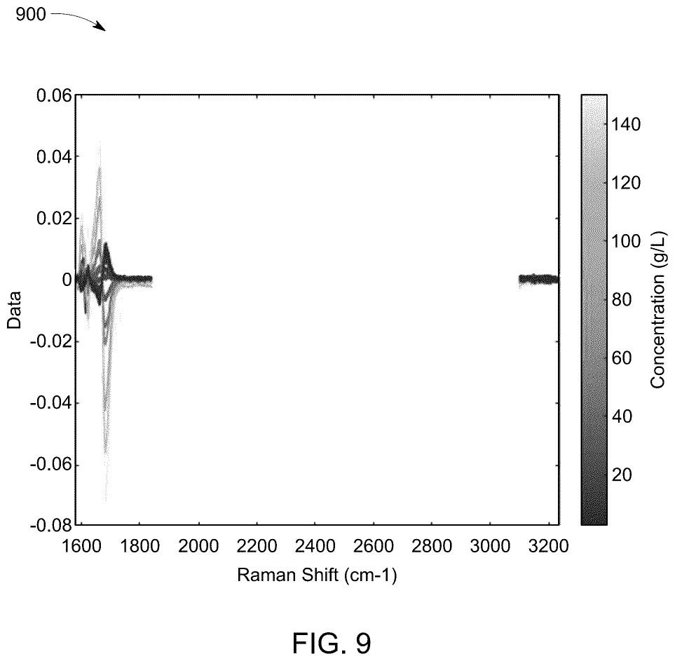

FIGS. 8-9 graphically illustrate several preprocessing operations applied to the set 700 according to some examples. These preprocessing operations can be performed, e.g., during the model calibration or in the block 504 of the method 500. The preprocessing operations illustrated in FIG. 8 include selecting two narrower spectral regions from the full spectral range of the set 700 (FIG. 7). A first selected spectral region 802 includes the wavenumber range between approximately 600 cm⁻¹ and approximately 1850 cm⁻¹, which covers the above-mentioned “Amide I” region of the vibrational spectra. A second selected spectral region 804 includes the wavenumber range between approximately 3140 cm⁻¹ and approximately 3260 cm⁻¹, which covers a corresponding water vibration band. For monochromatic green excitation light (e.g., having a wavelength of 532 nm), the wavenumber range of the spectral region 804 corresponds to red light to which the CCD detector 460 typically has relatively low sensitivity (quantum yield). As a result, only substances present in the sample at a relatively high concentration and/or having a relatively strong Raman activity can produce a prominent detectable Raman signal in this wavenumber range. Consequently, for aqueous solutions, the second selected spectral region 804 is dominated by the water signal (typically with no or minimum spectral interference). For different concentrations of the protein in the solution, the water concentration remains substantially constant, at 55.55 M. The latter characteristic is used during the model calibration or in the block 504 of the method 500 to normalize the detected Raman spectra and/or various spectral portions thereof.

A set 900 of the preprocessed spectra illustrated in FIG. 9 is obtained from the spectra illustrated in FIG. 8 by applying additional preprocessing operations that include: (i) infinity normalization using the water peak of the second selected spectral region 804 (FIG. 8); (ii) applying Savitzky-Golay filtering of the second order with a suitably selected sliding window width (e.g., thirteen data points); (iii) computing a first derivative of the filtered signal; and (iv) performing mean centering.

A Savitzky-Golay filter is a digital filter that can be applied to a set of digital data points for a purpose of smoothing the data without distorting the signal tendencies. This result is achieved, in a process known as convolution, by fitting successive sub-sets of adjacent data points with a low-degree polynomial by the method of linear least squares. When the data points are equally spaced, an analytical solution to the least-squares equations can be found and then used to give estimates of the smoothed signal and to compute derivatives of the smoothed signal. Numerical solutions can be found in other cases.

In various examples, the normalization operation calculates one of several different metrics using selected variables of each sample. Example options include: (i) 1-Norm; (ii) 2-Norm; and (iii) Infinity Norm. Computing the 1-Norm includes dividing each variable by the sum of absolute values of all selected variables for the given sample. The 1-Norm returns a vector with unit area (area=1) “under the curve.” Computing the 2-Norm includes normalizing to the square root of the sum of the squared values of all selected variables for the given sample. The 2-Norm returns a vector of unit length (length=1) and represents a form of weighted normalization where larger values are weighted more heavily in the scaling. Computing the Infinity Norm includes normalizing to the maximum value observed for all selected variables for the given sample. The Infinity Norm returns a vector with unit maximum value and represents a form of weighted normalization where only the largest value is considered in the scaling.

In some examples, the Infinity Norm is used to implement the normalization operations used in the above-mentioned preprocessing. Mathematically, the Infinity Norm is expressed as follows:

$${w}_{i} = MAX ( {x}_{i} ) \qquad ( 1 )$$

$${x}_{i , norm} = {x}_{i} {w}_{i}^{- 1} \qquad ( 2 )$$

where wᵢ is the normalization weight for sample i; and xᵢ is the vector of observed values for the given sample.

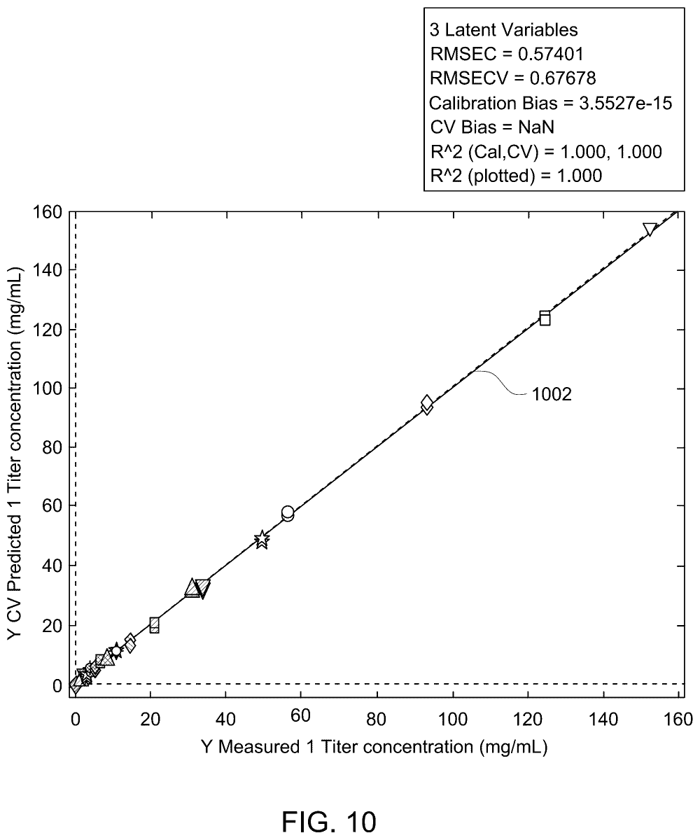

FIG. 10 graphically illustrates a fit 1002 to a set of calibration data, obtained with a partial least squares regression (PLS) statistical method, according to one example. In the example shown, the calibration data are acquired with the Raman instrument 400 (FIG. 4) connected to the Raman probe 300 (FIG. 3), which is operated at a flow rate of 100 mL/min. The following acquisition parameters are used: (i) excitation wavelength 785 nm; (ii) laser power 450 mW; (iii) integration time three seconds; (iv) average of three acquisitions; and (v) 10 replicates per concentration value. The acquired Raman spectra are subjected to preprocessing operations as described above in reference to FIG. 5 (block 504) and in further reference to FIGS. 7-9.

The fit 1002 is obtained by feeding the preprocessed calibration data into the Eigen Vector SOLO software configured under the PLS option. In other examples, other suitable commercially available software and/or other statistical-method options, such as principal component regression (PCR), least absolute shrinkage model and selection operator (LASSO), and elastic net regression, can alternatively be used. The optimized model parameters obtained during the fit are used to construct the above-mentioned first chemometric model. This model is then cross-validated using the leave-out-one method (contiguous block of 10). In other examples, different cross validation strategies, such as K-fold validation, random subsets, venetian blinds, and others, can be used. In some examples, a single chemometric model covering a relatively wide concentration range (e.g., 0 to 150 g/L) is constructed in this manner. In some other examples, a split chemometric model having two constituent sub-models for lower and higher concentrations, respectively, is similarly built. In some examples, the first sub-model is used to predict concentrations lower than 20 g/L, and the second sub-model is used to predict concentrations in the range from 20 g/L to 150 g/L.

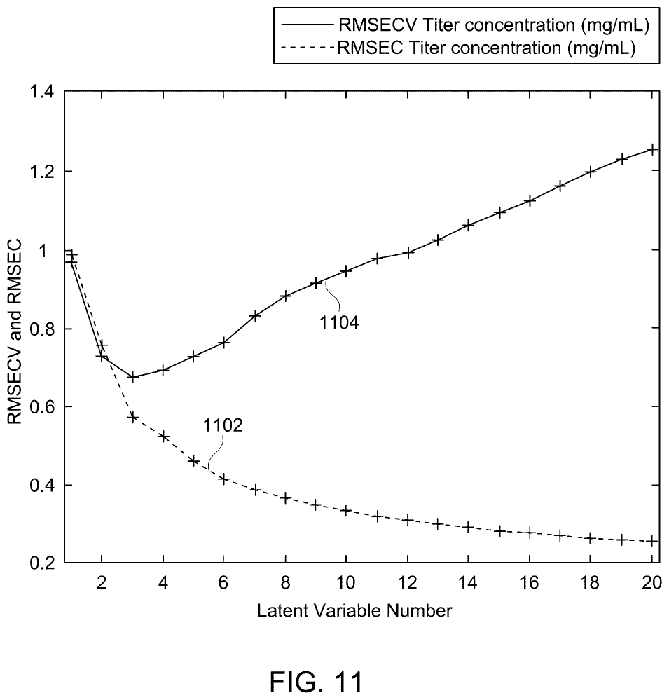

FIG. 11 graphically illustrates optimization of the above-described first chemometric model for the number of latent variables according to one example. In the example shown, the metrics used for the optimization are the root-mean-standard error for calibration (RMSEC) and the root-mean-standard error for cross-validation (RMSECV). Curves 1102 and 1104 shown in FIG. 11 plot the RMSEC and RMSECV values, respectively, as a function of the number of latent variables used in the first chemometric model. The curves 1102 and 1104 indicate that a preferred number of latent variables for the first chemometric model is 3 or 4. As indicated in the legend of FIG. 10, the fit 1002 corresponds to three latent variables. A comparably good fit (not shown) is obtained when four latent variables are used.

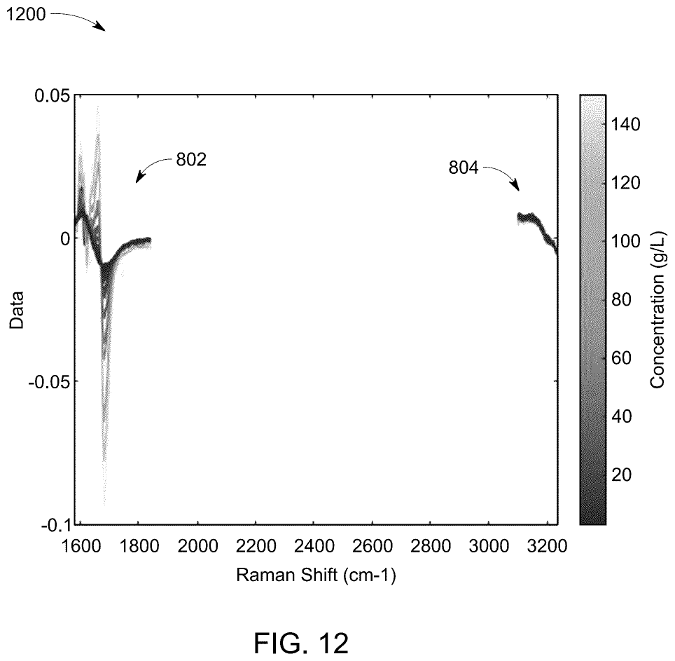

FIGS. 12-14 graphically illustrate selected features of the first chemometric model according to one example. More specifically, FIG. 12 graphically illustrates a set 1200 of preprocessed spectra obtained in one or more instances of the block 504 of the method 500 according to one example. The corresponding preprocessing operations include: (i) selecting the first and second spectral regions 802, 804 from the full spectral range of the acquired Raman spectra; (ii) normalizing the spectra based on the water peak located in the second selected spectral region 804; (iii) applying Savitzky-Golay filtering to the normalized spectra; and (iv) computing a first derivative of the resulting filtered signal.

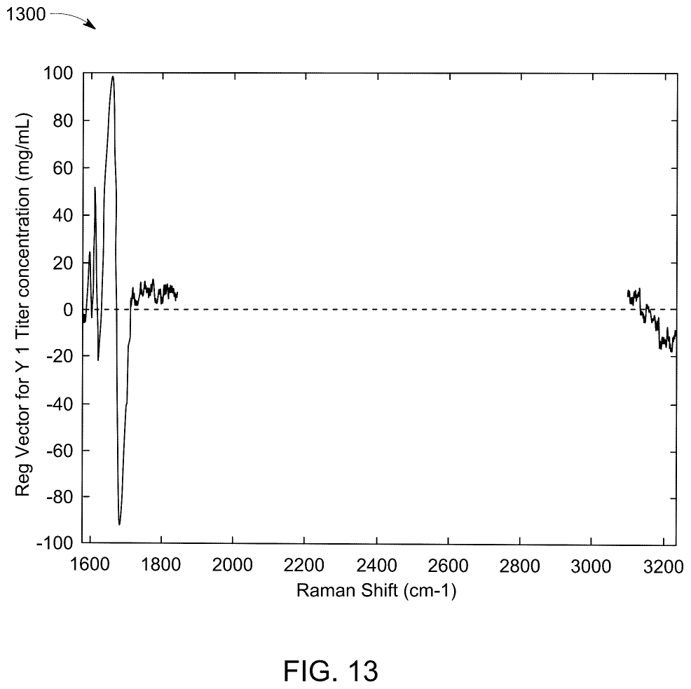

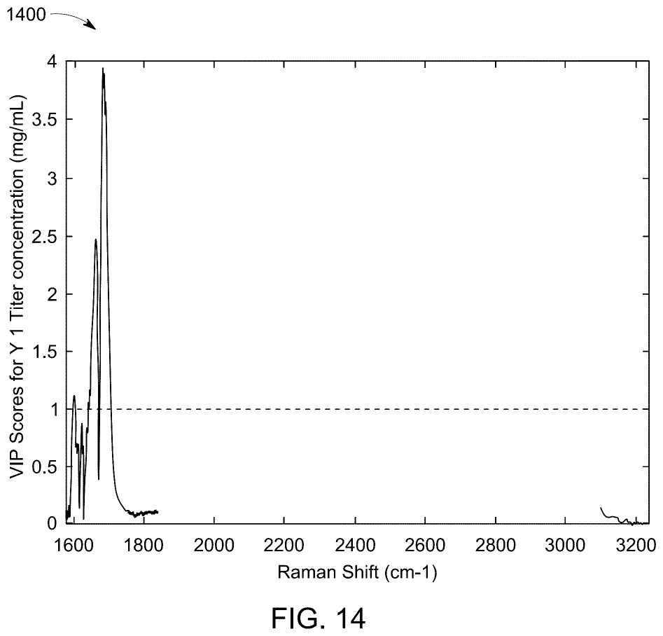

FIG. 13 graphically illustrates a regression vector 1300 computed at one of the operations of the block 508 of the method 500 using the first chemometric model constructed as described above and further using one of the preprocessed spectra from the set 1200 as an input to that model. FIG. 14 graphically illustrates the variable importance in projection (VIP) scores 1400 corresponding to the regression vector 1300. The VIP scores 1400 indicate that the first chemometric model for IgG is dominated by the Amide I region, which is beneficial for many intended applications of the model and/or method 500. For example, many monoclonal antibodies have the beta sheet as a dominant secondary structure. As already mentioned above, the spectral features of the Amide I region are directly relatable to the backbone conformation, which underscores the utility of the first chemometric model for quality control in the biopharmaceutical production process 100 configured to produce monoclonal antibodies.

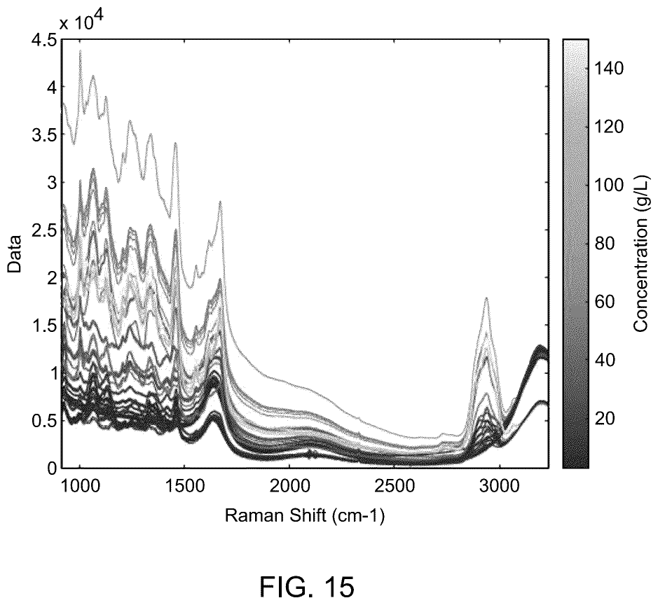

FIGS. 15-21 graphically illustrate a second chemometric model and its use in the method 500 according to some examples. The second chemometric model makes use of the Raman features spectrally located in the wavenumber range between approximately 900 cm⁻¹ and approximately 3300 cm⁻¹. This wavenumber range is wider than the wavenumber range used in the first chemometric model. As such, the second chemometric model can also be referred to as the “Extended Region” model.

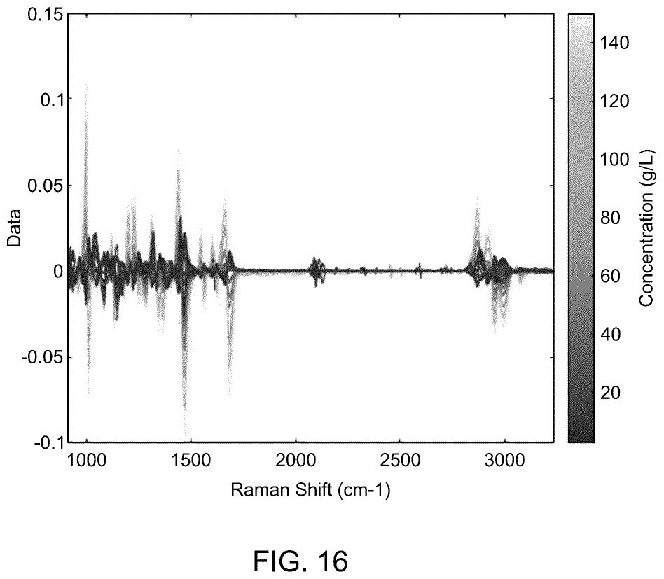

FIGS. 15-16 graphically illustrate several preprocessing operations applied to the set 700 according to some examples. These preprocessing operations can be performed, e.g., during the model calibration or in the block 504 of the method 500. The preprocessing operations illustrated in FIG. 15 include selecting a narrower spectral region from the full spectral range of the set 700 (FIG. 7). A set 1600 of the preprocessed spectra illustrated in FIG. 16 is obtained from the spectra illustrated in FIG. 15 by applying additional preprocessing operations that include: (i) infinity normalization using the water peak spectrally located at approximately 3200 cm⁻¹; (ii) applying Savitzky-Golay filtering of the second order with a sliding window width of thirteen data points; (iii) computing a first derivative of the filtered signal; and (iv) applying mean centering.

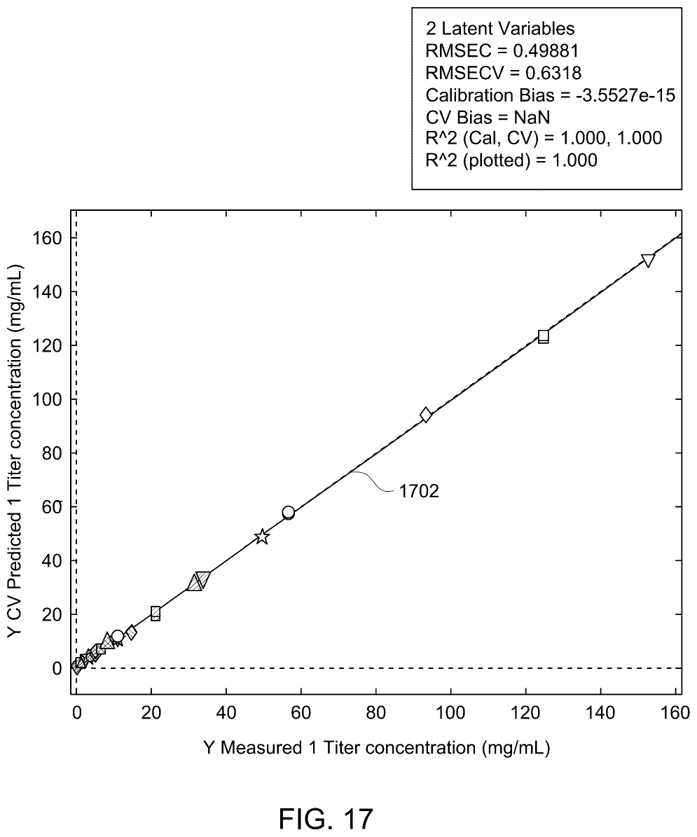

FIG. 17 graphically illustrates a fit 1702 to the above-described calibration data, obtained with a partial least squares regression (PLS) statistical method, according to one example. The fit 1702 is obtained by feeding the preprocessed calibration data into the Eigen Vector SOLO software configured under the PLS option. In other examples, other software and/or other statistical-method options, such as PCR, LASSO, and elastic net regression, can alternatively be selected. The optimized model parameters obtained during the fit are used to construct the above-mentioned second chemometric model. This model is then cross-validated using the leave-out-one method (contiguous block of 10). In some examples, a single chemometric model covering a relatively wide concentration range (e.g., 0 to 150 g/L) is constructed in this manner. In some other examples, a split chemometric model having two constituent sub-models for lower and higher concentrations is similarly built. In some examples, the first sub-model is used to predict concentrations lower than 20 g/L, and the second sub-model is used to predict concentrations in the range from 20 g/L to 150 g/L.

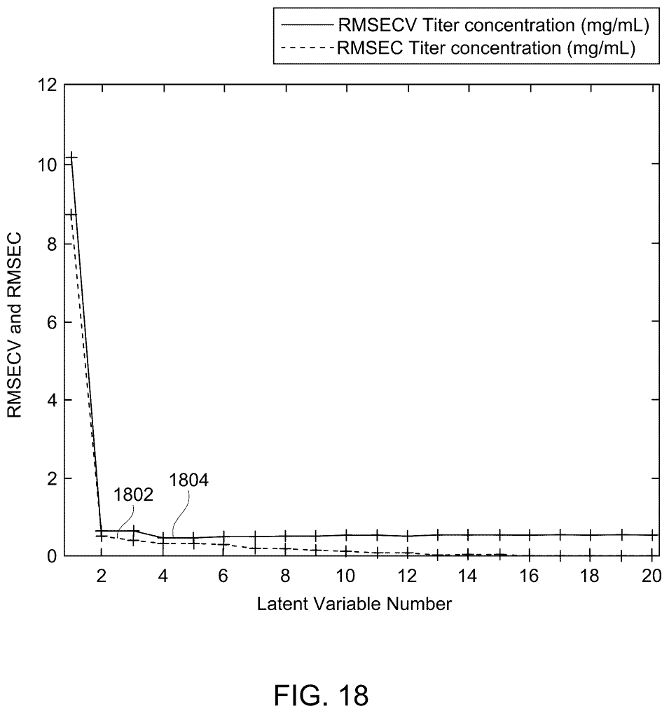

FIG. 18 graphically illustrates optimization of the above-described second chemometric model for the number of latent variables according to one example. In the example shown, the metrics used for the optimization are the RMSEC and the RMSECV. Curves 1802 and 1804 shown in FIG. 18 plot the RMSEC and RMSECV values, respectively, as a function of the number of latent variables used in the second chemometric model. The curves 1802 and 1804 indicate that a preferred number of latent variables for the second chemometric model is 2 or 3. As indicated in the legend of FIG. 17, the fit 1702 corresponds to two latent variables. A comparably good fit (not shown) is obtained when three latent variables are used.

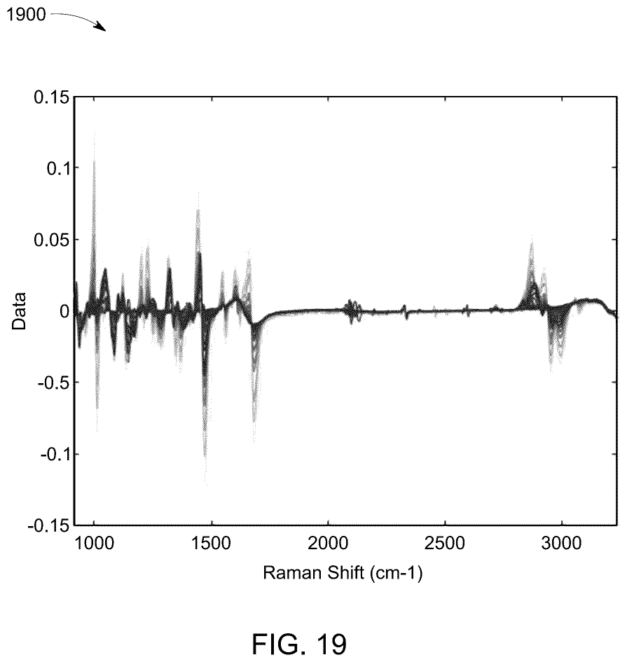

FIGS. 19-21 graphically illustrate selected features of the second chemometric model according to one example. More specifically, FIG. 19 graphically illustrates a set 1900 of preprocessed spectra obtained in one or more instances of the block 504 of the method 500 according to one example. The corresponding preprocessing operations include: (i) selecting the above-described narrower spectral region from the full spectral range of the acquired Raman spectra; (ii) normalizing the spectra based on the water peak spectrally located at approximately 3200 cm⁻¹; (iii) applying a Savitzky-Golay filter to the normalized spectra; and (iv) computing a first derivative of the resulting filtered signal.

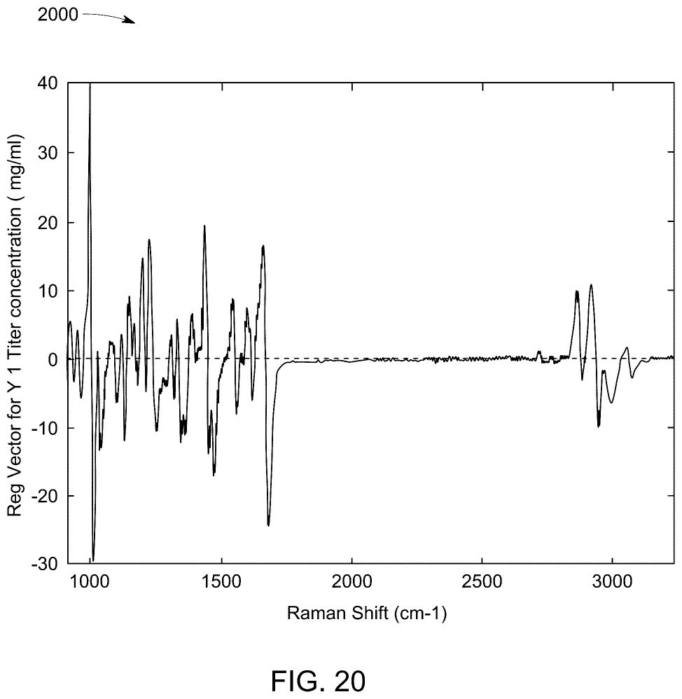

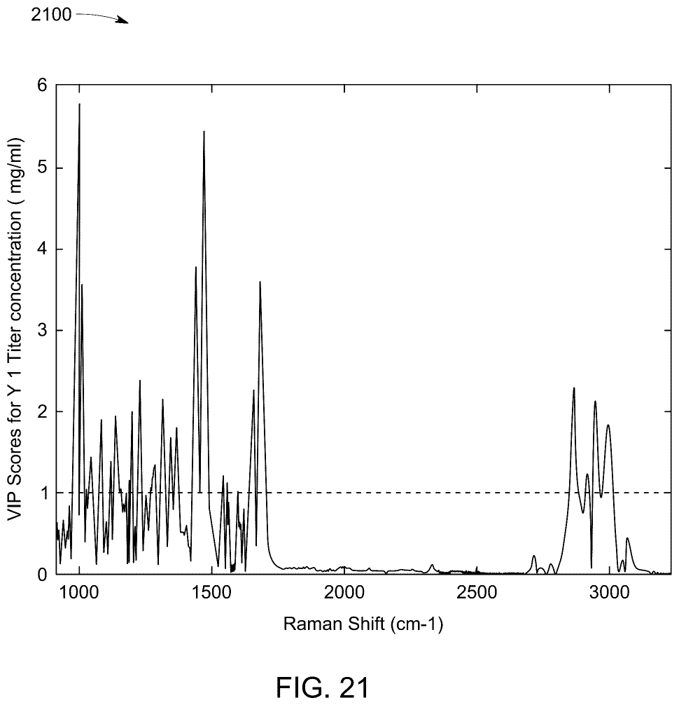

FIG. 20 graphically illustrates a regression vector 2000 computed at one of the operations of the block 508 of the method 500 using the second chemometric model constructed as described above and further using one of the preprocessed spectra from the set 1900 as an input to that model. FIG. 21 graphically illustrates the VIP scores 2100 corresponding to the regression vector 2000. The VIP scores 2100 indicate that the second chemometric model for IgG is dominated by the Amide I, CH-deformation, phenylalanine, and CH-stretching regions, which is beneficial for at least some intended applications of the model and/or method 500.

FIG. 22 graphically illustrates improvements in in situ protein concentration measurements obtained with the method 500 according to some examples. More specifically, three kinetics curves, labeled 2212, 2214, and 2216, respectively, are shown in FIG. 22. The kinetics curve 2212 is obtained using the method 500 employing the above-described first chemometric model. The kinetics curve 2214 is obtained using the method 500 employing the above-described second chemometric model. The kinetics curve 2216 is obtained using in-line UV-Vis measurements. Each of the kinetics curves 2212, 2214, 2216 spans three phases 2202, 2204, 2206 of the UF/DF process, which is a part of the block 160 of the biopharmaceutical production process 100 (also see FIGS. 1-2). The phase 2202 is a first product-concentration phase that takes place from time zero to time t₀. The phase 2204 is a diafiltration phase that takes place from time t₀ to time t₁. The phase 2206 is a second product-concentration phase that takes place from time t₁ to time t₂.

The prediction error from the in-line UV-Vis measurements represented by the kinetics curve 2216 is about 30% for the diafiltration phase 2204 and the second product-concentration phase 2206. In the example shown, the relatively large UV-Vis measurement error is caused by the spectral overlap between the used matrix and the protein absorbance caused by the aromatic amino acids (Tyrosine, Tryptophan, and Phenylalanine) thereof. In contrast, the Raman-based kinetics curves 2212, 2214 obtained with the method 500 as described above have an absolute error that is smaller than 10%. Both of the first and second chemometric models beneficially produce accurate and consistent concentration-measurement results that represent a clear improvement with respect to the UV-Vis measurements.

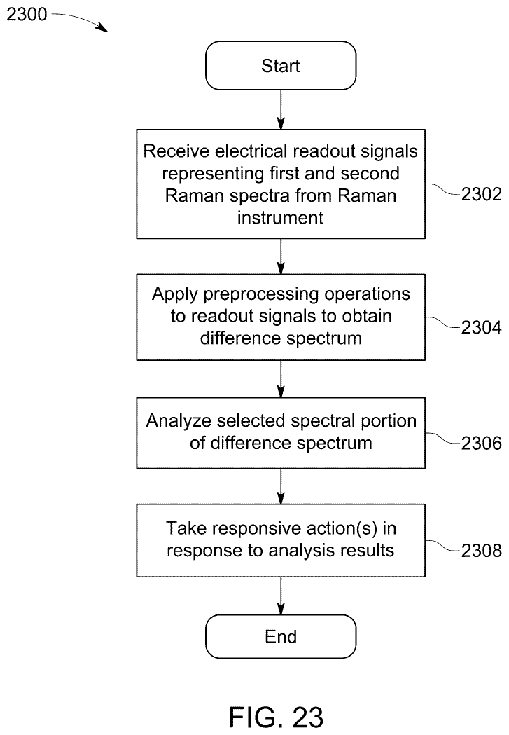

FIG. 23 is a flowchart illustrating a method 2300 performed via a computing device for providing support to the Raman instrument 400 according to further examples. In some examples, the method 2300 is used for real-time or near real-time protein secondary-structure elucidation. Determination of the secondary structure of the protein produced using the biopharmaceutical production process 100 provides information on the quality of the product, and appropriate adjustments to the process can be made to address any discovered quality issues. For example, denaturation, degradation, and/or aggregation of the protein may cause undesirable changes in the protein secondary structure, which are beneficially detectable with the method 2300.

The method 2300 includes the computing device receiving from the detector 460 of the Raman instrument 400 first and second electrical readout signals 462 (in a block 2302). The first readout signal 462 represents a first Raman spectrum acquired with the Raman instrument 400 from a product sample having a first concentration of the protein in question, e.g., of a monoclonal antibody. The second readout signal 462 represents a second Raman spectrum acquired with the Raman instrument 400 from a product sample having a second concentration of the protein in question. The second concentration is higher than the first concentration. For example, such first and second Raman spectra can be acquired at different respective times during the second concentration stage 2206 (FIG. 22) of the UF/DF process using the inline Raman probe 226ₙ inserted into the retentate return channel 256 of the system 200. A nonlimiting example of the first and second acquisition times t₁ and t₂ is indicated in FIG. 22 as an illustration. The first and second Raman spectra are typically acquired using the same set of acquisition parameters.

The method 2300 also includes the computing device applying a set of preprocessing operations (in a block 2304) to the first and second electrical readout signal(s) received in the block 2302. In various examples, the set of preprocessing operations of the block 2304 is directed at converting the first and second Raman spectra into a corresponding difference spectrum. In various examples, the set of preprocessing operations of the block 2304 includes: (i) excluding the spectral region below 300 cm⁻¹; (ii) applying Savitzky-Golay filtering; (iii) baseline removal; (iv) normalization; and (v) computing a difference spectrum of the first and second Raman spectra after those spectra are preprocessed via operations (i)-(iv). In some examples, some of the preprocessing operations of the block 2304 are implemented similar to the corresponding preprocessing operations of the block 504 of the method 500 (FIG. 5).

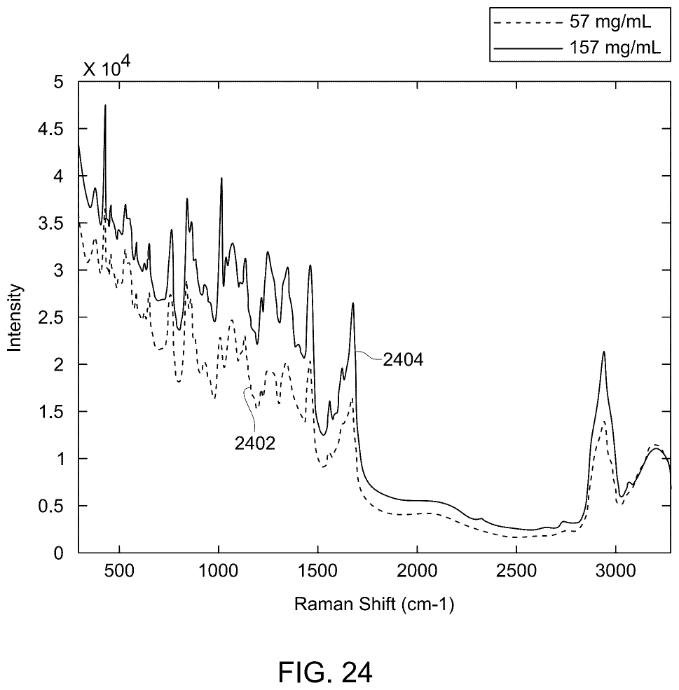

The method 2300 also includes the computing device analyzing (in a block 2306) a selected spectral portion of the difference spectrum obtained in the block 2304. In some examples, for the samples containing monoclonal antibodies, the selected spectral portion is in the wavenumber range between approximately 1500 cm⁻¹ and 1750 cm⁻¹. In other examples, other suitable spectral ranges can also be selected. In various examples, the block 2306 may include some or all of the following operations: (i) peak deconvolution; (ii) determination of spectral positions of the deconvoluted peaks; (iii) computation of the deconvoluted peak areas; (iv) mapping of the deconvoluted peaks onto the corresponding protein secondary-structure features; and (v) comparing the mapping results with applicable product specifications. Illustrative examples of the operations of the block 2306 are described in more detail below in reference to FIGS. 24-30.

The method 2300 also includes the computing device performing or initiating one or more responsive actions (in a block 2308). The responsive actions of the block 2308 are performed or initiated in response to the results of the analysis performed in the block 2306. One example of such responsive action includes the computing device displaying the secondary-structure mapping results of the block 2306 on a display device, e.g., using a GUI. Another example of such responsive action includes the computing device generating a visual or audible alert when the secondary-structure mapping results of the block 2306 place the tested product outside the acceptable margins defined in the applicable product specifications. In some cases, one or more of the responsive actions indicated in FIG. 6 can also be performed or initiated in the block 2308. Upon completion of the operations of the block 2308, the method 2300 is terminated.

FIGS. 24-30 graphically and schematically illustrate certain operations of the method 2300 according to some examples.

FIG. 24 graphically illustrates Raman spectra 2402, 2404 obtained in the block 2302 of the method 2300 according to one example. The Raman spectrum 2402 is an example of the above-described first Raman spectrum and corresponds to the protein concentration of 57 g/L. The Raman spectrum 2404 is an example of the above-described second Raman spectrum and corresponds to the protein concentration of 157 g/L.

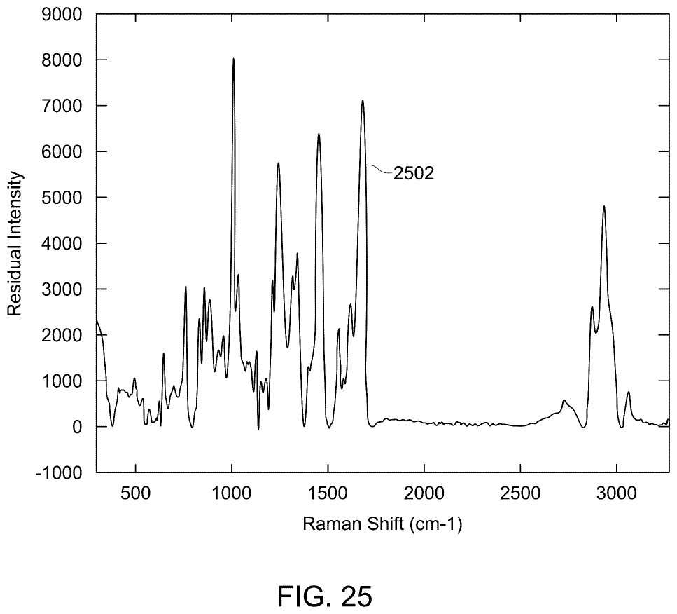

FIG. 25 graphically illustrates a difference spectrum 2502 obtained in the block 2304 of the method 2300 according to one example. More specifically, the difference spectrum 2502 is computed as described above based on the Raman spectra 2402, 2404 (FIG. 24).

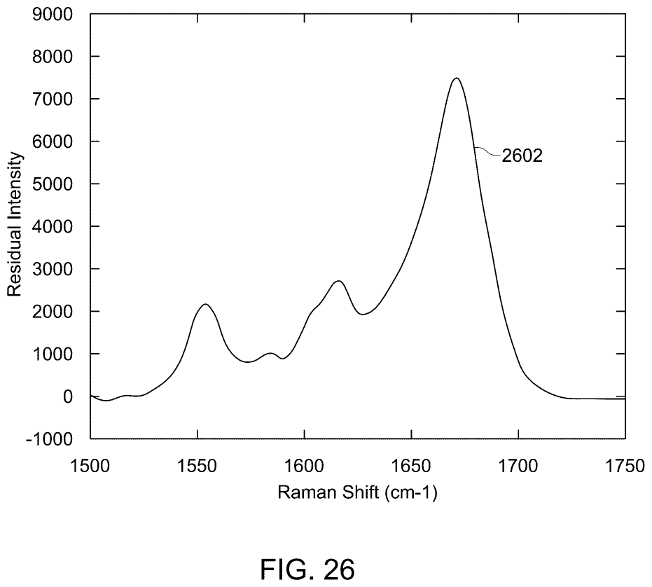

FIG. 26 graphically illustrates a spectral portion 2602 of the difference spectrum 2502 (FIG. 25) selected for analysis in the block 2306 of the method 2300 according to one example. The spectral portion 2602 covers the wavenumber region between 1500 cm⁻¹ and 1750 cm⁻¹. As already indicated above, in the case of monoclonal antibodies, this spectral region has spectral features that are directly relatable to the backbone conformation of the protein and are sensitive to the secondary structure.

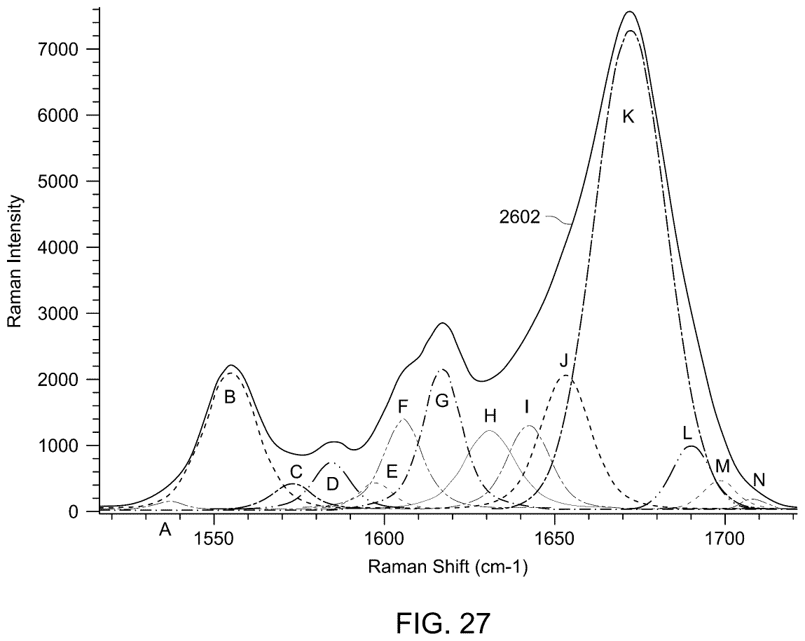

FIG. 27 graphically illustrates the peak deconvolution operation of the block 2306 of the method 2300 according to one example. In the example shown, individual constituent peaks of the spectral portion 2602 are identified using the second derivative of the spectral envelope. The Voigt function is then used to model each of the identified peaks, and the parameters of the Voigt functions are iteratively adjusted until the sum of Voigt functions provides a best fit to the shape of the spectral portion 2602. In the example shown, the deconvolution operation results in fourteen peaks, which are labeled A-N in FIG. 27. Each of the peaks A-N is characterized by a set of parameter values including: (i) the wavenumber corresponding to the center of the peak; (ii) the height of the peak; (iii) the peak's full width at half height or FWHH; and (iv) the peak area.

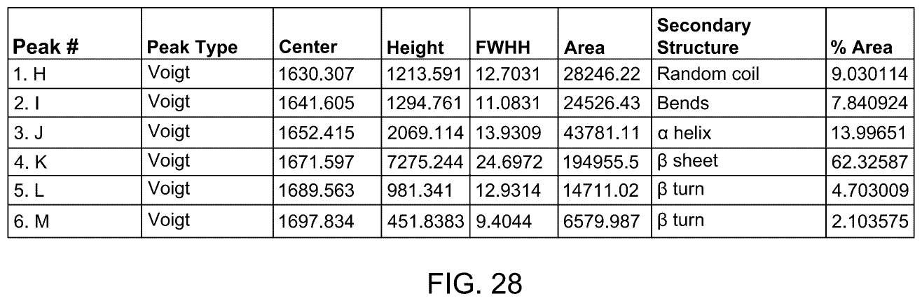

FIG. 28 shows a table listing a subset H-M of the peaks A-N determined via the deconvolution operation illustrated in FIG. 27 according to one example. For each of the peaks H-M, the table includes the respective set of parameter values mentioned above. The table also includes a mapping of each peak onto a corresponding element of the secondary structure and an estimate of its relative contribution based on the percentage with respect to the total area of the corresponding spectral envelope. Example elements of the secondary structure may include a random coil, a backbone bend, an alpha helix, a beta sheet, a beta turn, etc.

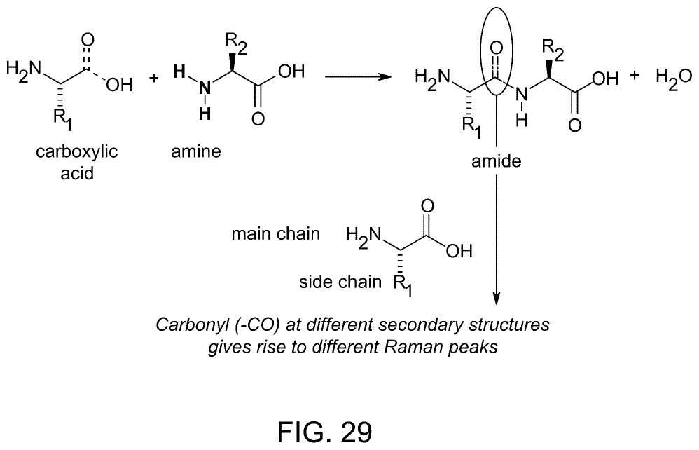

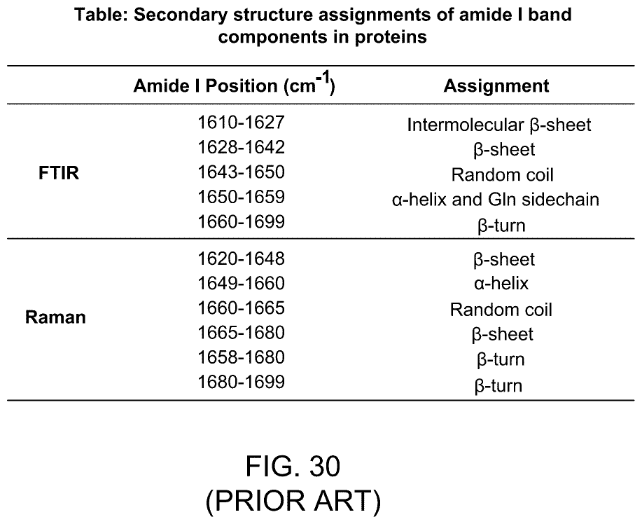

FIGS. 29-30 illustrate supporting information for the peak mappings to the elements of the secondary structure according to one example. This supporting information can be used, e.g., to generate the mapping presented in FIG. 28 in at least some examples. For example, FIG. 29 schematically illustrates the chemical basis of the sensitivity of the spectral features of the Amide I region to the secondary structure of the protein. As indicated in FIG. 29, the secondary structure affects the immediate environment of the carbonyl groups in the backbone, which manifests itself in different peak positions in the vibrational spectra. FIG. 30 provides example assignments of various peaks in the Raman and infrared spectra to specific elements of the secondary structure. In various additional examples, other types of supporting information can also be used to configure the mapping operations of the block 2306 of the method 2300.

FIG. 31 is a block diagram illustrating a computing device 3100 one or more instances of which can be used with the process 100 according to some examples. In various examples, one or more instances of the computing device 3100 can be used to control the equipment used in the biopharmaceutical production process 100 and/or to implement the methods 500 and 2300. In some examples, an instance of the computing device is used to implement an electronic controller of a piece of equipment used in the biopharmaceutical production process 100.

The computing device 3100 of FIG. 31 is illustrated as having a number of components, but any one or more of these components may be omitted or duplicated, as suitable for the application and setting. In some embodiments, some or all of the components included in the computing device 3100 may be attached to one or more motherboards and enclosed in a housing. In some embodiments, some of those components may be fabricated onto a single system-on-a-chip (SoC) (e.g., the SoC may include one or more electronic processing devices 3102 and one or more storage devices 3104). Additionally, in various embodiments, the computing device 3100 may not include one or more of the components illustrated in FIG. 31, but may include interface circuitry for coupling to the one or more components using any suitable interface (e.g., a Universal Serial Bus (USB) interface, a High-Definition Multimedia Interface (HDMI) interface, a Controller Area Network (CAN) interface, a Serial Peripheral Interface (SPI) interface, an Ethernet interface, a wireless interface, or any other appropriate interface). For example, the computing device 3100 may not include a display device 3110, but may include display device interface circuitry (e.g., a connector and driver circuitry) to which an external display device 3110 may be coupled.

The computing device 3100 includes a processing device 3102 (e.g., one or more processing devices). As used herein, the terms “electronic processor device” and “processing device” interchangeably refer to any device or portion of a device that processes electronic data from registers and/or memory to transform that electronic data into other electronic data that may be stored in registers and/or memory. In various embodiments, the processing device 3102 may include one or more digital signal processors (DSPs), application-specific integrated circuits (ASICs), central processing units (CPUs), graphics processing units (GPUs), server processors, or any other suitable processing devices.

The computing device 3100 also includes a storage device 3104 (e.g., one or more storage devices). In various embodiments, the storage device 3104 may include one or more memory devices, such as random-access memory (RAM) devices (e.g., static RAM (SRAM) devices, magnetic RAM (MRAM) devices, dynamic RAM (DRAM) devices, resistive RAM (RRAM) devices, or conductive-bridging RAM (CBRAM) devices), hard drive-based memory devices, solid-state memory devices, networked drives, cloud drives, or any combination of memory devices. In some embodiments, the storage device 3104 may include memory that shares a die with the processing device 3102. In such an embodiment, the memory may be used as cache memory and include embedded dynamic random-access memory (eDRAM) or spin transfer torque magnetic random-access memory (STT-MRAM), for example. In some embodiments, the storage device 3104 may include non-transitory computer readable media having instructions thereon that, when executed by one or more processing devices (e.g., the processing device 3102), cause the computing device 3100 to perform any appropriate ones of the methods disclosed herein below or portions of such methods.

The computing device 3100 further includes an interface device 3106 (e.g., one or more interface devices 3106). In various embodiments, the interface device 3106 may include one or more communication chips, connectors, and/or other hardware and software to govern communications between the computing device 3100 and other computing devices. For example, the interface device 3106 may include circuitry for managing wireless communications for the transfer of data to and from the computing device 3100. The term “wireless” and its derivatives may be used to describe circuits, devices, systems, methods, techniques, communications channels, etc., that may communicate data via modulated electromagnetic radiation through a nonsolid medium. The term does not imply that the associated devices do not contain any wires, although in some embodiments they might not. Circuitry included in the interface device 3106 for managing wireless communications may implement any of a number of wireless standards or protocols, including but not limited to Institute for Electrical and Electronic Engineers (IEEE) standards including Wi-Fi (IEEE 802.11 family), IEEE 802.16 standards, Long-Term Evolution (LTE) project along with any amendments, updates, and/or revisions (e.g., advanced LTE project, ultramobile broadband (UMB) project (also referred to as “3GPP2”), etc.). In some embodiments, circuitry included in the interface device 3106 for managing wireless communications may operate in accordance with a Global System for Mobile Communication (GSM), General Packet Radio Service (GPRS), Universal Mobile Telecommunications System (UMTS), High Speed Packet Access (HSPA), Evolved HSPA (E-HSPA), or LTE network. In some embodiments, circuitry included in the interface device 3106 for managing wireless communications may operate in accordance with Enhanced Data for GSM Evolution (EDGE), GSM EDGE Radio Access Network (GERAN), Universal Terrestrial Radio Access Network (UTRAN), or Evolved UTRAN (E-UTRAN). In some embodiments, circuitry included in the interface device 3106 for managing wireless communications may operate in accordance with Code Division Multiple Access (CDMA), Time Division Multiple Access (TDMA), Digital Enhanced Cordless Telecommunications (DECT), Evolution-Data Optimized (EV-DO), and derivatives thereof, as well as any other wireless protocols that are designated as 3G, 4G, 5G, and beyond. In some embodiments, the interface device 3106 may include one or more antennas (e.g., one or more antenna arrays) configured to receive and/or transmit wireless signals.

In some embodiments, the interface device 3106 may include circuitry for managing wired communications, such as electrical, optical, or any other suitable communication protocols. For example, the interface device 3106 may include circuitry to support communications in accordance with Ethernet technologies. In some embodiments, the interface device 3106 may support both wireless and wired communication, and/or may support multiple wired communication protocols and/or multiple wireless communication protocols. For example, a first set of circuitry of the interface device 3106 may be dedicated to shorter-range wireless communications such as Wi-Fi or Bluetooth, and a second set of circuitry of the interface device 3106 may be dedicated to longer-range wireless communications such as global positioning system (GPS), EDGE, GPRS, CDMA, WiMAX, LTE, EV-DO, or others. In some other embodiments, a first set of circuitry of the interface device 3106 may be dedicated to wireless communications, and a second set of circuitry of the interface device 3106 may be dedicated to wired communications.

The computing device 3100 also includes battery/power circuitry 3108. In various embodiments, the battery/power circuitry 3108 may include one or more energy storage devices (e.g., batteries or capacitors) and/or circuitry for coupling components of the computing device 3100 to an energy source separate from the computing device 3100 (e.g., to AC line power).

The computing device 3100 also includes a display device 3110 (e.g., one or multiple individual display devices). In various embodiments, the display device 3110 may include any visual indicators, such as a heads-up display, a computer monitor, a projector, a touchscreen display, a liquid crystal display (LCD), a light-emitting diode display, or a flat panel display.

The computing device 3100 also includes additional input/output (I/O) devices 3112. In various embodiments, the I/O devices 3112 may include one or more data/signal transfer interfaces, audio I/O devices (e.g., microphones or microphone arrays, speakers, headsets, earbuds, alarms, etc.), audio codecs, video codecs, printers, sensors (e.g., thermocouples or other temperature sensors, humidity sensors, pressure sensors, vibration sensors, etc.), image capture devices (e.g., one or more cameras), human interface devices (e.g., keyboards, cursor control devices, such as a mouse, a stylus, a trackball, or a touchpad), etc.

Depending on the specific embodiment, various components of the interface devices 3106 and/or I/O devices 3112 can be configured to output suitable control signals, receive suitable control/telemetry signals, and receive and transmit data streams. In some examples, the interface devices 3106 and/or I/O devices 3112 include one or more analog-to-digital converters (ADCs) for transforming received analog signals into a digital form suitable for operations performed by the processing device 3102 and/or the storage device 3104. In some additional examples, the interface devices 3106 and/or I/O devices 3112 include one or more digital-to-analog converters (DACs) for transforming digital signals provided by the processing device 3102 and/or the storage device 3104 into an analog form suitable for being transmitted through a communication channel.

According to one example disclosed above, e.g., in the summary section and/or in reference to any one or any combination of some or all of FIGS. 1-31, provided is a method performed via a computing device for providing support to a Raman spectrometry (RS) system, the method comprising: receiving from the RS system a set of electrical readout signals representing a Raman spectrum of a sample including a protein; applying a set of preprocessing operations to the Raman spectrum to obtain a corresponding preprocessed Raman spectrum conforming to an input format of a selected multivariate chemometric model; and estimating a concentration of the protein in the sample using the selected multivariate chemometric model and the preprocessed Raman spectrum.

In some examples of the above method, the sample includes a volume of fluid flowing through a flow-cell Raman probe connected to equipment used in a biopharmaceutical production process configured to make or purify the protein.

In some examples of any of the above methods, the sample includes a volume of fluid adjacent to an immersible Raman probe placed in a bioreactor or product holding vessel of equipment used in a biopharmaceutical production process configured to make or purify the protein.

In some examples of any of the above methods, the equipment is configured to implement a stage of the biopharmaceutical production process selected from the group consisting of: centrifugation and microfiltration operations of upstream processing; ultrafiltration of the upstream processing; capture chromatography of downstream processing; viral inactivation of the downstream processing; intermediate and polishing chromatography of the downstream processing; viral filtration of the downstream processing; ultrafiltration and diafiltration of the downstream processing; bulk fill operations; and dispense fill operations.

In some examples of any of the above methods, the set of preprocessing operations includes normalization of the Raman spectrum based on an intensity of a water vibration band thereof.

In some examples of any of the above methods, the water vibration band has a maximum in a wavenumber range between 3140 cm⁻¹ and 3260 cm⁻¹.

In some examples of any of the above methods, the set of preprocessing operations further includes one or more operations selected from the group consisting of: averaging two or more of the readout signals; baseline removal; spectrum smoothing; computing a derivative of a smoothed spectrum; exclusion of one or more wavenumber ranges; and mean centering.

In some examples of any of the above methods, the exclusion operation comprises removing from consideration spectral data located in a wavenumber range between 1900 cm⁻¹ and 3000 cm⁻¹ or between 1850 cm⁻¹ and 3050 cm⁻¹.

In some examples of any of the above methods, the exclusion operation comprises removing from consideration spectral data located in a wavenumber range below 900 cm⁻¹ or below 950 cm⁻¹.

In some examples of any of the above methods, the selected multivariate chemometric model is constructed using calibration data and a statistical method selected from the group consisting of: partial least squares regression (PLS); principal component regression (PCR); least absolute shrinkage model and selection operator (LASSO); and elastic net regression.

In some examples of any of the above methods, the method further comprises: selecting a first multivariate chemometric model when an expected concentration value is greater than a threshold value; and selecting a second multivariate chemometric model when the expected concentration value is smaller than the threshold value.

In some examples of any of the above methods, the input format of the first multivariate chemometric model has spectral data located in a wavenumber range between 1900 cm⁻¹ and 3000 cm⁻¹ or between 1850 cm⁻¹ and 3050 cm⁻¹ excluded from consideration; and wherein the input format of the second multivariate chemometric model has spectral data located in a wavenumber range below 900 cm⁻¹ or below 950 cm⁻¹ excluded from consideration.

In some examples of any of the above methods, the first multivariate chemometric model has a first number of latent variables; and wherein the second multivariate chemometric model has a different second number of latent variables.

In some examples of any of the above methods, the selected multivariate chemometric model is trained with calibration data corresponding to a first protein; and wherein the sample includes a different second protein.

In some examples of any of the above methods, the selected multivariate chemometric model is trained with calibration data obtained with samples of the protein in a first buffer; and wherein the sample includes a different second buffer.

In some examples of any of the above methods, the method further comprises performing or initiating a responsive action based on the estimated concentration.

In some examples of any of the above methods, the responsive action is selected from the group consisting of a process information action; a release action; an in-process control action; and an equipment control action.

In some examples of any of the above methods, the protein is a monoclonal antibody.

A non-transitory computer-readable medium storing instructions that, when executed by the computing device, cause the computing device to perform operations comprising any one of the above methods.

According to another example disclosed above, e.g., in the summary section and/or in reference to any one or any combination of some or all of FIGS. 1-31, provided is an apparatus comprising: a Raman spectrometry (RS) system; and a computing device configured to: receive from the RS system a set of electrical readout signals representing a Raman spectrum of a sample including a protein; apply a set of preprocessing operations to the Raman spectrum to obtain a corresponding preprocessed Raman spectrum conforming to an input format of a selected multivariate chemometric model; and estimate a concentration of the protein in the sample using the selected multivariate chemometric model and the preprocessed Raman spectrum.

In some examples of the above apparatus, the computing device is further configured to perform or initiate a responsive action based on the estimated concentration.

In some examples of any of the above apparatus, the RS system comprises a Raman probe configured to be coupled to biopharmaceutical production equipment used to make or purify the protein.

In some examples of any of the above apparatus, the Raman probe includes a flow cell device inserted into a line in the biopharmaceutical production equipment carrying a fluid containing the protein between a first equipment unit and a second equipment unit.

In some examples of any of the above apparatus, the Raman probe is an immersible probe placed into a bioreactor or product holding vessel having a fluid containing the protein, the bioreactor or product holding vessel being a part of the biopharmaceutical production equipment.

In some examples of any of the above apparatus, the biopharmaceutical production equipment is used for upstream processing.

In some examples of any of the above apparatus, the biopharmaceutical production equipment is used for downstream processing.

In some examples of any of the above apparatus, the computing device is further configured to perform or initiate a control action based on the estimated concentration, the control action being directed at the biopharmaceutical production equipment and selected form the group consisting of: a process information action; a release action; an in-process control action; and an equipment control action.

According to yet another example disclosed above, e.g., in the summary section and/or in reference to any one or any combination of some or all of FIGS. 1-31, provided is a method performed via a computing device for providing support to a Raman spectrometry (RS) system, the method comprising: receiving from the RS system a set of electrical readout signals representing a first Raman spectrum and a second Raman spectrum of first and second samples, respectively, including a protein in different respective concentrations; applying a set of preprocessing operations to the first and second Raman spectra to obtain a corresponding difference spectrum; and analyzing a selected spectral portion of the difference spectrum to evaluate a secondary structure of the protein.

In some examples of the above method, each of the first and second samples includes a volume of fluid flowing through a flow-cell Raman probe connected to equipment used in a biopharmaceutical production process configured to make or purify the protein.

In some examples of any of the above methods, each of the first and second samples includes a volume of fluid adjacent to an immersible Raman probe placed in a bioreactor or product holding vessel of equipment used in a biopharmaceutical production process configured to make or purify the protein.

In some examples of any of the above methods, the set of preprocessing operations includes normalization of the first and second Raman spectra based on respective intensities of a water vibration band thereof.

In some examples of any of the above methods, the water vibration band has a maximum in a wavenumber range between 3140 cm⁻¹ and 3260 cm⁻¹.

In some examples of any of the above methods, the set of preprocessing operations further includes one or more operations selected from the group consisting of: averaging two or more of the readout signals; baseline removal; spectrum smoothing; and exclusion of one or more wavenumber ranges.

In some examples of any of the above methods, the selected spectral portion includes a wavenumber range between 1520 cm⁻¹ and 1720 cm⁻¹.

In some examples of any of the above methods, the analyzing comprises one or more operations selected from the group consisting of: peak deconvolution; determination of spectral positions of deconvoluted peaks; computation of peak areas of the deconvoluted peaks; mapping the deconvoluted peaks to corresponding protein secondary-structure features; and comparing the mapping with product specifications.

In some examples of any of the above methods, the method further comprises performing or initiating a responsive action based on the evaluation.

In some examples of any of the above methods, the responsive action is selected from the group consisting of a process information action; a release action; an in-process control action; and an equipment control action.

In some examples of any of the above methods, the protein is a monoclonal antibody.

A non-transitory computer-readable medium storing instructions that, when executed by the computing device, cause the computing device to perform operations comprising any one of the above methods.

According to yet another example disclosed above, e.g., in the summary section and/or in reference to any one or any combination of some or all of FIGS. 1-31, provided is an apparatus comprising: a Raman spectrometry (RS) system; and a computing device configured to: receive from the RS system a set of electrical readout signals representing a first Raman spectrum and a second Raman spectrum of first and second samples, respectively, including a protein in different respective concentrations; apply a set of preprocessing operations to the first and second Raman spectra to obtain a corresponding difference spectrum; and analyze a selected spectral portion of the difference spectrum to evaluate a secondary structure of the protein.

In some examples of the above apparatus, the computing device is further configured to perform or initiate a responsive action based on the evaluation.

In some examples of any of the above apparatus, the RS system comprises a Raman probe configured to be coupled to biopharmaceutical production equipment used to make or purify the protein.

In some examples of any of the above apparatus, the Raman probe includes a flow cell device inserted into a line in the biopharmaceutical production equipment carrying a fluid containing the protein between a first equipment unit and a second equipment unit.

In some examples of any of the above apparatus, the Raman probe is an immersible probe placed into a bioreactor or product holding vessel having a fluid containing the protein, the bioreactor or product holding vessel being a part of the biopharmaceutical production equipment.

In some examples of any of the above apparatus, the biopharmaceutical production equipment is used for upstream processing.

In some examples of any of the above apparatus, the biopharmaceutical production equipment is used for downstream processing.

In some examples of any of the above apparatus, the computing device is further configured to perform or initiate a control action based on the evaluation, the control action being directed at the biopharmaceutical production equipment and selected form the group consisting of: a process information action; a release action; an in-process control action; and an equipment control action.

It is to be understood that the above description is intended to be illustrative and not restrictive. Many implementations and applications other than the examples provided would be apparent upon reading the above description. The scope should be determined, not with reference to the above description, but should instead be determined with reference to the appended claims, along with the full scope of equivalents to which such claims are entitled. It is anticipated and intended that future developments will occur in the technologies discussed herein, and that the disclosed systems and methods will be incorporated into such future examples. In sum, it should be understood that the application is capable of modification and variation.

All terms used in the claims are intended to be given their broadest reasonable constructions and their ordinary meanings as understood by those knowledgeable in the technologies described herein unless an explicit indication to the contrary is made herein. In particular, use of the singular articles such as “a,” “the,” “said,” etc. should be read to recite one or more of the indicated elements unless a claim recites an explicit limitation to the contrary.

The Abstract is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various examples for the purpose of streamlining the disclosure. This method of disclosure is not to be interpreted as reflecting an intention that the claimed subject matter incorporate more features than are expressly recited in each claim. Rather, as the following claims reflect, inventive subject matter lies in fewer than all features of a single disclosed example. Thus, the following claims are hereby incorporated into the Detailed Description, with each claim standing on its own as a separately claimed subject matter.

Unless explicitly stated otherwise, each numerical value and range should be interpreted as being approximate as if the word “about” or “approximately” preceded the value or range.

Although the elements in the following method claims, if any, are recited in a particular sequence with corresponding labeling, unless the claim recitations otherwise imply a particular sequence for implementing some or all of those elements, those elements are not necessarily intended to be limited to being implemented in that particular sequence.

Unless otherwise specified herein, the use of the ordinal adjectives “first,” “second,” “third,” etc., to refer to an object of a plurality of like objects merely indicates that different instances of such like objects are being referred to, and is not intended to imply that the like objects so referred-to have to be in a corresponding order or sequence, either temporally, spatially, in ranking, or in any other manner.

Unless otherwise specified herein, in addition to its plain meaning, the conjunction “if”' may also or alternatively be construed to mean “when” or “upon” or “in response to determining” or “in response to detecting,” which construal may depend on the corresponding specific context. For example, the phrase “if it is determined” or “if [a stated condition] is detected” may be construed to mean “upon determining” or “in response to determining” or “upon detecting [the stated condition or event]” or “in response to detecting [the stated condition or event].”

Also for purposes of this description, the terms “couple,” “coupling,” “coupled,” “connect,” “connecting,” or “connected” refer to any manner known in the art or later developed in which energy is allowed to be transferred between two or more elements, and the interposition of one or more additional elements is contemplated, although not required. Conversely, the terms “directly coupled,” “directly connected,” etc., imply the absence of such additional elements.

The functions of the various elements shown in the figures, including any functional blocks labeled as “processors” and/or “controllers,” may be provided through the use of dedicated hardware as well as hardware capable of executing software in association with appropriate software. When provided by a processor, the functions may be provided by a single dedicated processor, by a single shared processor, or by a plurality of individual processors, some of which may be shared. Moreover, explicit use of the term “processor” or “controller” should not be construed to refer exclusively to hardware capable of executing software, and may implicitly include, without limitation, digital signal processor (DSP) hardware, network processor, application specific integrated circuit (ASIC), field programmable gate array (FPGA), read only memory (ROM) for storing software, random access memory (RAM), and nonvolatile storage. Other hardware, conventional and/or custom, may also be included. Similarly, any switches shown in the figures are conceptual only. Their function may be carried out through the operation of program logic, through dedicated logic, through the interaction of program control and dedicated logic, or even manually, the particular technique being selectable by the implementer as more specifically understood from the context.

As used in this application, the terms “circuit,” “circuitry” may refer to one or more or all of the following: (a) hardware-only circuit implementations (such as implementations in only analog and/or digital circuitry); (b) combinations of hardware circuits and software, such as (as applicable): (i) a combination of analog and/or digital hardware circuit(s) with software/firmware and (ii) any portions of hardware processor(s) with software (including digital signal processor(s)), software, and memory(ies) that work together to cause an apparatus, such as a mobile phone or server, to perform various functions); and (c) hardware circuit(s) and or processor(s), such as a microprocessor(s) or a portion of a microprocessor(s), that requires software (e.g., firmware) for operation, but the software may not be present when it is not needed for operation.” This definition of circuitry applies to all uses of this term in this application, including in any claims. As a further example, as used in this application, the term circuitry also covers an implementation of merely a hardware circuit or processor (or multiple processors) or portion of a hardware circuit or processor and its (or their) accompanying software and/or firmware. The term circuitry also covers, for example and if applicable to the particular claim element, a baseband integrated circuit or processor integrated circuit for a mobile device or a similar integrated circuit in server, a cellular network device, or other computing or network device.

It should be appreciated by those of ordinary skill in the art that any block diagrams herein represent conceptual views of illustrative circuitry embodying the principles of the disclosure. Similarly, it will be appreciated that any flow charts, flow diagrams, state transition diagrams, pseudo code, and the like represent various processes which may be substantially represented in computer readable medium and so executed by a computer or processor, whether or not such computer or processor is explicitly shown.

What is claimed is:

1. An apparatus, comprising:
a Raman spectrometry (RS) system; and
a computing device configured to:
receive from the RS system a set of electrical readout signals representing a Raman spectrum of a sample including a protein;
apply a set of preprocessing operations to the Raman spectrum to obtain a corresponding preprocessed Raman spectrum conforming to an input format of a selected multivariate chemometric model; and
estimate a concentration of the protein in the sample using the selected multivariate chemometric model and the preprocessed Raman spectrum.

2. The apparatus of claim 1, wherein the computing device is further configured to perform or initiate a responsive action based on the estimated concentration.

3. The apparatus of claim 1, wherein the RS system comprises a Raman probe configured to be coupled to biopharmaceutical production equipment used to make or purify the protein.

4. The apparatus of claim 3, wherein the Raman probe includes a flow cell device inserted into a line in the biopharmaceutical production equipment carrying a fluid containing the protein between a first equipment unit and a second equipment unit.

5. The apparatus of claim 3, wherein the Raman probe is an immersible probe placed into a bioreactor or product holding vessel having a fluid containing the protein, the bioreactor or product holding vessel being a part of the biopharmaceutical production equipment.

6. The apparatus of claim 3, wherein the computing device is further configured to perform or initiate a control action based on the estimated concentration, the control action being directed at the biopharmaceutical production equipment and selected form the group consisting of:
a process information action;
a release action;
an in-process control action; and
an equipment control action.

7. A method performed via a computing device for providing support to a Raman spectrometry (RS) system, the method comprising:
receiving from the RS system a set of electrical readout signals representing a Raman spectrum of a sample including a protein;
applying a set of preprocessing operations to the Raman spectrum to obtain a corresponding preprocessed Raman spectrum conforming to an input format of a selected multivariate chemometric model; and
estimating a concentration of the protein in the sample using the selected multivariate chemometric model and the preprocessed Raman spectrum.

8. The method of claim 7, wherein the sample includes a volume of fluid flowing through a flow-cell Raman probe connected to equipment used in a biopharmaceutical production process configured to make or purify the protein, and wherein the equipment is configured to implement a stage of the biopharmaceutical production process selected from the group consisting of:
centrifugation and microfiltration operations of upstream processing;
ultrafiltration of the upstream processing;
capture chromatography of downstream processing;
viral inactivation of the downstream processing;
intermediate and polishing chromatography of the downstream processing;
viral filtration of the downstream processing;
ultrafiltration and diafiltration of the downstream processing;
bulk fill operations; and
dispense fill operations.

9. The method of claim 7, wherein the set of preprocessing operations includes normalization of the Raman spectrum based on an intensity of a water vibration band thereof.

10. The method of claim 9, wherein the water vibration band has a maximum in a wavenumber range between 3140 cm⁻¹ and 3260 cm⁻¹.

11. The method of claim 7, wherein the set of preprocessing operations further includes one or more operations selected from the group consisting of:
averaging two or more of the readout signals;
baseline removal;
spectrum smoothing;
computing a derivative of a smoothed spectrum;
exclusion of one or more wavenumber ranges; and
mean centering.

12. The method of claim 7, wherein the selected multivariate chemometric model is constructed using calibration data and a statistical method selected from the group consisting of:
partial least squares regression (PLS);
principal component regression (PCR);
least absolute shrinkage model and selection operator (LASSO); and
elastic net regression.

13. The method of claim 7, further comprising:
selecting a first multivariate chemometric model when an expected concentration value is greater than a threshold value; and
selecting a second multivariate chemometric model when the expected concentration value is smaller than the threshold value.

14. The method of claim 13,
wherein the input format of the first multivariate chemometric model has spectral data located in a wavenumber range between 1900 cm⁻¹ and 3000 cm⁻¹ or between 1850 cm⁻¹ and 3050 cm⁻¹ excluded from consideration; and
wherein the input format of the second multivariate chemometric model has spectral data located in a wavenumber range below 900 cm⁻¹ or below 950 cm⁻¹ excluded from consideration.

15. The method of claim 13,
wherein the first multivariate chemometric model has a first number of latent variables; and
wherein the second multivariate chemometric model has a different second number of latent variables.

16. The method of claim 7,
wherein the selected multivariate chemometric model is trained with calibration data corresponding to a first protein; and
wherein the sample includes a different second protein.

17. The method of claim 7,
wherein the selected multivariate chemometric model is trained with calibration data obtained with samples of the protein in a first buffer; and
wherein the sample includes a different second buffer.

18. The method of claim 7, further comprising performing or initiating a responsive action based on the estimated concentration.

19. The method of claim 18, wherein the responsive action is selected from the group consisting of:
a process information action;
a release action;
an in-process control action; and
an equipment control action.

20. A non-transitory computer-readable medium storing instructions that, when executed by a computing device, cause the computing device to perform operations comprising:
receiving from a Raman spectrometry (RS) system a set of electrical readout signals representing a Raman spectrum of a sample including a protein;
applying a set of preprocessing operations to the Raman spectrum to obtain a corresponding preprocessed Raman spectrum conforming to an input format of a selected multivariate chemometric model; and
estimating a concentration of the protein in the sample using the selected multivariate chemometric model and the preprocessed Raman spectrum.
